(19) United States
(12) Patent Application Publication
(10) Pub. No.: US 2010/0028263 A1
(43) Pub. Date: Feb. 4, 2010

(54) Methods for inhibiting mast cell activation and treating mast cell-dependent inflammatory diseases and disorders using lactobacillus

(75) Inventors: Cecile Schiffer-Mannioui, Paris (FR); Marc Daeron, Chevreuse (FR); Pierre Bruhns, Paris (FR)

Correspondence Address: OBLON, SPIVAK, MCCLELLAND MAIER & NEUSTADT, L.L.P., 1940 DUKE STREET, ALEXANDRIA, VA, 22314, US

(73) Assignees: INSTITUT PASTEUR, Paris (FR); INSTITUT NATIONAL DE LA SANTE ET, Paris (FR)

(21) Appl. No.: 12/461,137

(22) Filed: Aug. 3, 2009

Related U.S. Application Data
(60) Provisional application No. 61/085,680, filed on Aug. 1, 2008.

(51) Int. Cl. A61K 49/00; A61K 35/74; A01K 67/00; C12N 5/00; A61P 29/00

(52) U.S. Cl. 424/9.2; 424/93.45; 800/9; 435/375

(57) ABSTRACT

Lactobacillus compositions, especially those based on L. casei, and methods for the prevention and treatment of diseases or disorders involving or mediated by mast cells, such as anaphylaxis, allergy, autoimmune and inflammatory disorders including arthritis and rheumatoid arthritis.

### CROSS-REFERENCE TO RELATED APPLICATIONS

This application claims priority under 35 U.S.C. §119(e) to U.S. Provisional Application No. 61/085,680, filed Aug. 1, 2008, and to the corresponding PCT ______, filed ______, 2009, both of which are incorporated by reference.

### BACKGROUND OF THE INVENTION

1. Field of the Invention

The present invention pertains to Lactobacillus compositions and methods for the prevention and treatment of diseases or disorders involving mast cells, such as anaphylaxis, allergy, auto immune and inflammatory disorders including arthritis and rheumatoid arthritis.

2. Description of the Related Art

The recognition of a correlation between the decreasing incidence of infectious diseases and the increasing incidence of immune diseases over the second half of the 20^{th} century was at the origin of the hygiene hypothesis. According to this hypothesis, a lower exposure of the immune system to pathogens resulting from a better hygiene of life, the generalization of vaccination and an extensive use of antibiotics in developed countries, favored the development of Th2 responses at the expenses of Th1 responses. On the basis of this rationale, the hygiene hypothesis has been proposed to account for the allergy “epidemics” that has been expanding over the last decades. Noticeably, however, not only Th2-type diseases, such as allergies, but also Th1-type diseases, such as type-1 diabetes, Crohn's disease and multiple sclerosis have increased in parallel. A common feature of these two types of diseases is inflammation. Rather than mechanisms that differentially control Th cell polarization at the initiation of immune responses, mechanisms that control inflammation at the effector phase of immune responses may be affected by microorganisms.

Immune responses have been understood as resulting from a cooperation between the innate and the adaptive immune systems. As they express “pattern recognition” receptors for microorganisms or their soluble products, the many cells of the innate immune system can be activated by bacteria and viruses and contribute to the defense against pathogens. These cells also critically control adaptive immunity, and at two distinct steps. As dendritic cell activation by microbial products is pivotal in antigen presentation to T cells, the innate immune system controls the initiation of adaptive immune responses. It also controls the effector phase of adaptive immune responses. Most cells of the innate immune system indeed express receptors for the Fc portion of antibodies (FcRs). When binding to FcRs, antibodies provide these cells with bona fide antigen receptors, and enroll cells of the innate immunity into adaptive immunity. Besides endowing them with specificity, FcRs can indeed generate intracellular signals which modulate, positively or negatively, their biologic activities.

Mast cells are such cells. Their protective role against bacteria was dramatically demonstrated in the murine model of peritonitis induced by cecal ligation and puncture (Echtenacher et al., 1996) and in experimental Klebsiella pneumoniae infection (Thakurdas et al., 2007). They are also critical in inflammation. Mast cells express high-affinity IgE receptors (FcεRI), and their role in the initiation of allergic reactions is well known. They also express low-affinity IgG receptors. These include activating FcRs (FcγRIIIA in mice or FcγRIIA in humans) and inhibitory FcRs (FcγRIIB in both species), the engagement of which by immune complexes controls mast cell responses. Mast cells were recently found to critically determine IgG-dependent tissue inflammation in autoimmune diseases. They have been implicated in rheumatoid arthritis, Sjögren's syndrome, systemic sclerosis, multiple sclerosis and thyroiditis in humans, and in experimental allergic encephalomyelitis, in the K/B×N model of rheumatoid arthritis (Lee et al, Science 2002), in a bullous pemphigoid-like disease and in a model of glomerulonephritis in mice. Mast cells can therefore control the expression not only of Th2-, but also of Th1-type inflammatory diseases.

Some probiotic strains have already been described as having a beneficial effect on some inflammatory diseases, including certain allergies. However, all the experiments published to date focus on the induction of the immune response, leading to allergies or other inflammatory diseases. For example, Kim et al. describe that oral probiotic bacterial administration to mice during 7 weeks, starting 2 weeks before the initial sensitization by ovalbumin, suppress the induction of allergic responses in this model (Kim et al., 2005).

However, immune responses such as allergic responses can be divided in two phases, namely the induction phase and the effector phase. To date, nothing is known about the effect of probiotic strains on the effector phase (which necessitates mast cell activation).

### BRIEF SUMMARY OF THE INVENTION

As exemplified in the experimental part below, the inventors surprisingly demonstrated that some probiotics are able to inhibit mast cell activation, thereby having protective effects against certain human inflammatory diseases, including autoimmune diseases and allergies. Importantly, the results obtained by the inventors show that these probiotics can prevent pathogenic immune responses even in subjects who have already been sensitized or who have already developed an auto-immune disease. This opens a new therapeutic window for these probiotics, since patients who already have developed an inflammatory disease can be treated according to the invention described below.

The present invention pertains to a process for treating a disease or disorder mediated by mast cells comprising administering to a mammal in need thereof, or contacting the mast cells of a mammal with, a composition comprising a lactic acid bacterium, Lactobacillus or a component of Lactobacillus. Lactic acid bacteria and Lactobacillus strains include L. casei, such as a preferred strain L. casei DN 114-001 (also called L. casei defensis), deposited at the CNCM (Collection Nationale de Culture de Microorganismes, 25 rue du Docteur Roux, Paris) under I-1518 on Dec. 30, 1994. Bifidobacterium breve strains, including the strain deposited at the CNCM under the number 1-2219 on May 31, 1999, may also be mentioned. These compositions may also include other strains from the genera Lactobacillus, Lactococcus and Streptococcus or other microorganisms conventionally used to produce fermented foods such yogurt, yogurt-like drinks, probiotic food products, kefir and cheeses.

These bacterial strains may be used in the form of intact, living bacteria or as dead or inactivated bacteria, such as whole irradiated or whole chemically-treated bacteria. Alternatively, lysates or fractions of these bacteria which modulate mast cell activity and attenuate mast cell mediated diseases, conditions or disorders may be used. Such lysates or fractions may contain cytoplasmic, membrane, or peptidoglycan fractions, soluble or insoluble fractions, or specific protein-, lipid- or carbohydrate-containing compounds.

Compositions containing these bacteria or their lysates or fractions may be administered as food supplements, such as components or additives to a food, or in forms such as capsules, tablets, powders, troches, or other non-food associated forms.

This process may be used to treat an inflammatory disease or disorder mediated by mast cells by modulating mast cell activity. Such diseases and disorders include IgE-induced allergic conditions, an IgG-induced autoimmune disease or disorder, and arthritis.

This subject mammal may be a human, non-human primate, domestic pet animal, such as a dog or cat, a livestock animal such as a cow, sheep, goat, horse or pig, or other domesticated mammals suffering from a disease or disorder mediated by mast cells or at risk of developing such a disorder, such as mammals subject to vaccination, pharmaceutical treatment, or exposed to environmental allergens.

The Lactobacillus composition may be administered orally or parenterally either before or after the beginning or an inflammatory disease or disorder. Other routes of administration, such as topical, ocular, nasal, or inhalational administration or administration onto a mucus membrane, are contemplated which would result in exposure of mast cells to Lactobacillus or their components.

This process may also be performed by contacting the mast cells of the mammal with Lactobacillus or a component thereof ex vivo or in vitro.

Another aspect of the present invention is a screening process for identifying a bacterial composition, for example a Lactobacillus composition which prevents or treats a disease or disorder mediated by mast cells, comprising:

a) administering an amount of bacterial composition to a normal mouse by parenteral route,

b) subsequently injecting intravenously the serum from an autoimmune K/B×N mouse; and

c) determining the severity of arthritis in said normal mouse, optionally by analyzing the score of arthritis by clinical examination. A Lactobacillus such as L. casei may be used in this method. Subcellular components or fractions of Lactobacillus or inactivated Lactobacilli may also be screened using this method.

The invention also relates to a process of screening for identifying a bacterial composition, for example a Lactobacillus composition which treats or prevents a mast cell dependant allergic process comprising:

a) administering orally or parenterally of a bacterial composition to normal mice,

b) subsequently injecting intravenously said normal mice with purified IgE or serum containing IgE antibodies specific for an allergen,

c) challenging intravenously said IgE-injected mice, optionally one or more days later, with said allergen,

e) determining the intensity anaphylactic shock induced by said challenge, optionally by measuring a decrease in body temperature,

wherein a reduced anaphylactic shock compared to control mice not administered said bacterial composition is indicative of a bacterial composition which treats or prevents a mast cell dependant allergic process. L. casei may be employed in this method.

A composition comprising a Lactobacillus strain which regulates and/or controls in vivo, in mammals, the induction of an inflammation state involving mast cells, is also part of the present invention. This composition may also contain one or more additional biological or pharmaceutical components that modulate or mast cell activity such as histamines or anti-histamines, proteoglycans such as heparin, serine proteases, eicosanoids, such as prostaglandins and leukotrienes, or agents which stabilize mast cell membranes; or their antagonists. Pharmaceutical agents which regulate vasodilation and the associated sudden drop in blood pressure, edema, such as edema of the bronchial mucosa, and bronchoconstriction may also be coadminstered separately or in combination with the Lactobacillus composition.

The present invention also pertains to a model for anaphylaxis reaction, obtained by the following process:

a) sensitizing in vitro mast cells by contacting them with IgE specific for an allergen of interest,

b) injecting the sensitized mast cells to normal mice, subsequently

c) injecting into said mice the allergen of interest.

This model can be used for screening molecules likely to decrease the anaphylaxis shock, by administering these molecules to the animal either before step c) or after the beginning of the reaction. The intensity of the anaphylactic shock can optionally be monitored by measuring the body temperature of the mice injected with the allergen of interest.

Alternatively, the molecules to be tested can be incubated with the mast cells prior to step a). Accordingly, another aspect of the present invention is a process for identifying or screening a molecule which decreases the anaphylactic reaction in vivo, comprising:

a) pre-incubating mast cells with a molecule of interest,

b) sensitizing in vitro mast cells by contacting them with IgE specific for an allergen of interest,

c) injecting the sensitized mast cells to normal mice, subsequently

d) injecting into said mice the allergen of interest,

e) monitoring the anaphylactic shock, optionally by measuring the body temperature of said mice injected with the allergen of interest;

wherein a reduced amount of anaphylactic reaction in the treated mice compared to control mice administered mast cells not treated with said molecule of interest indicative of molecule which decreases the anaphylactic reaction in vivo. This process may be used to screen various biological, chemical or pharmaceutical molecules, including components of Lactobacillus including cytoplasmic molecules, peptidoglycan, or components of the Lactobacillus membrane.

The allergen of interest may be one commonly associated with anaphylaxis or allergy in humans, such as protein or polysaccharide allergens, and pharmaceutical products including antibiotics, biologicals including antibodies, serum, blood products and vaccines, cosmetic components, or allergens such as food allergens, such as milk, egg, cereal, peanut, soya, certain fruits and vegetables, or crustacean food products, animal dander, saliva, or hair, bee venom, dust mite or cockroach components, mold components or plant components, such as grass or tree pollen, or chemical agents, such as insecticides, herbicides, or petroleum or diesel products.

### BRIEF DESCRIPTION OF THE DRAWINGS

FIGS. 1(a)-(g). L. casei inhibits mast cell activation. (a) Inhibition of IgE-induced mast cell activation by live L. casei. BMMC were incubated overnight with PBS or live L. casei (100 bacteria/cell), sensitized with IgE anti-DNP and challenged with DNP-BSA. β-hexosaminidase was measured using an enzymatic colometric assay in BBMC supernatants, 20 min after challenge with the indicated concentrations of DNP-BSA. TNF-α was titrated using a cytotoxicity assay in BMMC supernatants 3 h after challenge with 10 ng/ml DNP-BSA. (b) Inhibition of IgE-induced mast cell activation by γ-irradiated L. casei. BMMC were incubated overnight with PBS or irradiated L. casei (1,000 bacteria/cell), sensitized and challenged as in FIG. 1a. (c) Time-dependent inhibition by γ-irradiated L. casei. BMMC were incubated with γ-irradiated L. casei for the indicated times, sensitized and challenged as in FIG. 1b. (d) Mast cell viability after exposure to γ-irradiated L. casei. BMMC incubated overnight with PBS, γ-irradiated L. casei or, as appositive control, with staurosporin were labeled with propidium iodide and allophycocyanin-conjugated annexin V, and analyzed by flow cytometry. (e) Inhibition is transient. BMMC incubated overnight with PBS or L. casei, were sensitized and challenged immediately or after the indicated times. (f) Inhibition of IgG-induced mast cell activation by γ-irradiated L. casei. PCMC were incubated overnight with PBS or γ-irradiated L. casei, sensitized and challenged as in FIG. 1b, or not sensitized and challenged with preformed immune GPI-a-GPI immune complexes. β-hexosaminidase was measured in supernatant 20 min later. (g) Inhibition of ionophore-induced mast cell activation by γ-irradiated L. casei. BMMC incubated overnight with PBS or γ-irradiated L. casei, sensitized and challenged as in FIG. 1b, were stimulated with thapsigargin or PMA and ionomycin. TNF-α was measured in supernatant 3 h later.

FIGS. 2(a)-(d). L. casei interacts with mast cells via an unknown receptor. (a) Inhibition of IgE-induced mast cell activation by γ-irradiated L. casei is decreased but not abrogated when cells and bacteria are separated by a membrane. BMMC were incubated overnight with PBS or live L. casei in a regular well in a Transwell (TW) (pore size: 0.4 μm), sensitized and challenged as in FIG. 1b. β-hexosaminidase and TNF-α were measured in supernatant as in FIG. 1a. (b) Interactions between mast cells and γ-irradiated L. casei. BMMC incubated overnight with FITC-labeled γ-irradiated L. casei as in FIG. 1b were stained with SNARF and examined by confocal video microscopy (the video of the same field is shown in FIG. 9). (c) Binding of γ-irradiated L. casei to mast cells. BMMC were incubated with FITC-labeled γ-irradiated L. casei for 45 min at 0° C. or at 37° C., treated with trypan blue or without, and fluorescence was monitored by flow cytometry. (d) Inhibition of IgE-induced activation of mast cells deficient in various receptors by γ-irradiated L. casei. BMMC from TLR2/4-, TLR3-, NOD2-, or MyD88-deficient mice were incubated overnight with PBS or γ-irradiated L. casei, sensitized with IgE and challenged with antigen as in FIG. 1b. β-hexosaminidase and TNF-α were measured in supernatant.

FIG. 3. L. casei is not phagocytosed by mast cells and does not activate mast cells. (A) BMMC or THP-1 cells were incubated with live L. casei, treated or not with gentamycin and lysed with TX-100. Lysates were plated onto MRS. (B) BMMC were sensitized with IgE and challenged with antigen, or incubated with the indicated ratios of L. casei/cell for 20 min (left panel) or 3 h (right panel). β-hexosaminidase and TNF-α were measured in supernatants as described in FIG. 1a legend. (C) BMMC were incubated with PBS, L. casei, S. thermophilus or 0.1 M phorbol myristic acetate (PMA)+1 μM ionomycin for 3 hours. Cytokines were measured in supernatants by the Luminex technology. * Below the detection level.

FIGS. 4(a)-(d). L. casei inhibits IgE-induced adoptive and passive systemic anaphylaxis. (a, b) Adoptive systemic anaphylaxis. 5×10⁶ BMMC previously incubated overnight with PBS or irradiated L. casei and sensitized with IgE anti-DNP (mAb 2682I) were injected i.v. to C57BL/6 mice. Mice were challenged with DNP-BSA i.v. 15 min later. (a) survival curves; (b) decrease of body temperature as a function of time in individual mice in the same experiment. (c, d) Passive systemic anaphylaxis. (c) C57BL/6 mice were injected i.p. with PBS or 5×10⁷ live L. casei at days −2 and −1. (d) C57BL/6 mice were given live L. casei in drinking water for 3 weeks and by daily gavage during the 4th week. Mice were injected i.v. with IgE anti-DNP (mAb SP20) at day −1 and challenged by an i.v. injection of DNP-BSA at day 0. The figure shows the mean decrease of body temperatures as a function of time (n=5). p values (Student's t test) of data from PBS- or L. casei-treated groups are indicated.

FIGS. 5(a), (b), (c) and (d). L. casei inhibits IgG-induced passive arthritis. (a) Inhibition of K/B×N serum-induced arthritis by L. casei i.p. C57BL/6 mice were injected i.p. daily with PBS or 5×10⁷ live L. casei from day −2 to day +4. They were injected i.v. with K/B×N serum at day 0. Arthritis was evaluated by measuring clinical index (see Methods). Mean clinical score (n=5) is shown as a function of time. (b) Inhibition of K/B×N serum-induced arthritis by L. casei administered i.p. or orally. C57BL/6 mice were either injected i.p. daily with PBS or 5×10⁷ live L. casei for 10 days, or given PBS or live L. casei in drinking water for 4 weeks and by gavage during the 4th week. They were injected i.v. with K/B×N serum, and Arthritis was evaluated by measuring clinical index (see Methods). Mean clinical score (n=5) is shown as a function of time. (c) Inhibition of K/B×N serum-induced arthritis by L. casei administered i.p. before or after disease induction. C57BL/6 mice were injected i.p. daily with PBS or 5×10⁷ live L. casei either from day −3 to day +2 or from day +2 to day +7. They were injected i.v. with K/B×N serum at day 0. Arthritis was evaluated by measuring clinical index (see Methods). Mean clinical score (n=5) is shown as a function of time. p values (Student's t test) of data from PBS- or L. casei-treated groups are indicated. (d) C57BL/6 mice, injected i.p. daily with PBS or L. casei between day −9 and +2, were injected i.v. with K/B×N serum at day 0. Ankle joint histology was analyzed at day +6.

FIGS. 6(a) and (b). L. casei inhibits human mast cell and basophil activation. (a) Inhibition of Ca2+ response in HMC-1 cells. HMC-1 cells were loaded with Fluo-3 and challenged with ionomycin. Relative intracellular Ca²⁺ concentration was monitored by flow cytometry as a function of time. (b, c) Inhibition of anti-IgE-induced basophil activation. Red cell-depleted blood cells from normal donors were incubated overnight with PBS or increasing numbers of γ-irradiated L. casei (b) or with PBS or 1,000 γ-irradiated L. casei/cell (c), and incubated with F(ab′)₂ fragments of anti-human IgE antibodies. Basophils, identified as FcεRI⁺, CD203⁺ cells, were gated, and CD203 expression was monitored in gated cells, before and after stimulation.

FIG. 7. Dose-dependent inhibition of IgE-induced mast cell activation by live and γ-irradiated L. casei. BMMC were incubated overnight with PBS or the indicated numbers of live L. casei/cell (left panel) or with PBS or the indicated numbers of γ-irradiated L. casei/cell (right panel). They were sensitized with IgE anti-DNP and challenged with DNP-BSA. β-hexosaminidase and TNF-α were measured in supernatant, 20 min and 3 h after stimulation, respectively.

FIGS. 8(a) and (b). Effects of three commensal bacteria on mast cells and on IgE-induced mast cell activation. (a) Inhibition of IgE-induced mast cell activation. BMMC were incubated overnight with PBS, or γ-irradiated L. casei, B. breve or S. Thermophilus, sensitized with IgE and challenged with antigen. γ-hexosaminidase and TNF-α were measured in supernatant, 20 min and 3 h after stimulation, respectively. (b) Mast cell activation. BMMC were incubated for 20 min or 3 h with PBS, or γ-irradiated L. casei, B. breve or S. Thermophilus. β-hexosaminidase and TNF-α were measured in supernatant, 20 min and 3 h after stimulation, respectively.

FIG. 9. Interactions of mast cells with γ-irradiated L. casei. BMMC incubated overnight with FITC-labeled γ-irradiated L. casei were stained with SNARF and examined by confocal video microscopy. Left image: same field as shown in FIG. 2b. Right image: another field in the same cell suspension.

FIG. 10. L. casei inhibits IgE-induced passive systemic anaphylaxis. C57BL/6 mice were injected with PBS or 5×10⁷ live L. casei i.p. from day −2 to day −1. They were injected with IgE anti-ovalbumin i.v. at day −1 and with ovalbumin at day 0. The figure shows the mean decrease of body temperatures as a function of time (n=5). p values (Student's t test) of data from PBS- or L. casei-treated groups are indicated.

FIG. 11. L. casei but not S. thermophilus inhibits IgE-induced mast cell activation. (A) BMMC were incubated overnight with PBS, live L. casei or live S. thermophilus, sensitized with IgE and challenged with antigen. β-hexosaminidase and TNF-α were measured in supernatants as described above. (B) BMMC were incubated overnight with PBS, or irradiated L. casei, sensitized with IgE and challenged with antigen. β-hexosaminidase and TNF-α were measured in supernatants as described above. (C) BMMC were incubated overnight with PBS, irradiated L. casei or irradiated S. thermophilus, sensitized with IgE and challenged with antigen. β-hexosaminidase and TNF-α were measured in supernatants as described above.

FIG. 12. L. casei inhibits IgE and IgG-induced mast cell activation. (A) BMMC were incubated overnight with PBS, live L. casei or live S. thermophilus, sensitized with IgE and challenged with antigen. Cytokines were measured in supernatants using the luminex technology in BMMC supernatants, 3 h after challenge with 10 ng/ml DNP-BSA. (B) PCMC were incubated overnight with PBS or live L. casei, sensitized with IgE and challenged with antigen (left panel), or challenged with preformed IgG immune complexes (right panel). β-hexosaminidase was measured in supernatants as described in FIG. 11A legend.

FIG. 13. L. casei inhibits IgE-induced intracellular signaling. (A) Schematic representation of major signaling pathways activated upon FcεRI engagement in mast cells. (B) BMMC incubated overnight with PBS or live L. casei were sensitized with IgE, loaded with Fluo-3-AM, and analyzed by flow cytometry before and after stimulation with antigen.

FIG. 14. Functional comparison of dendritic cells and mast cells at the induction phase and at the effector phase of immune responses. Antigen presentation by dendritic cells leads to the activation and differentiation of naive CD4 T cells into different T cell subsets. Mast cell activation by antibodies and antigens leads to the recruitment and activation of inflammatory cells. Both processes are amplified by pathogens and dampened by commensals.

### DETAILED DESCRIPTION OF THE PREFERRED EMBODIMENTS

The inventors have found that mast cells contribute to mechanisms underlying the hygiene hypothesis. The inventors have found that a commensal bacteria, Lactobacillus casei, profoundly inhibits IgE-induced and IgG-induced mast cell activation in vitro, IgE-induced passive systemic anaphylaxis and IgG-induced passive arthritis in the K/B×N model in vivo. L. casei also inhibited IgE-dependent human basophil activation. These findings expand on the general understanding of the hygiene hypothesis.

L. casei and other commensal bacteria have long been known to exert anti-inflammatory effects. These were observed in a variety of human inflammatory diseases and in murine models of allergy and autoimmunity. Studies on the mechanisms involved demonstrated that these bacteria affected TLR-mediated dendritic cell-dependent T cell activation and polarization, leading to alterations of the Th1/Th2 balance and the induction of regulatory T cells. Nonpathogenic bacteria can therefore affect the afferent phase of pro-inflammatory immune responses. The inventors show here that L. casei also profoundly affects the effector phase of immunologic inflammation.

L. casei indeed protected mice from IgE-dependent passive systemic anaphylaxis and from IgG-dependent passive arthritis i.e. in models which bypassed the induction phase of immune responses. Both models were demonstrated to depend on mast cells. Likewise, adoptive anaphylaxis induced by IgE-sensitized mast cells, was abrogated if mast cells were previously exposed to L. casei. Mast cell responses were indeed markedly inhibited by L. casei. Inhibition affected both degranulation and cytokine secretion, both IgE- and IgG-dependent responses, in both undifferentiated and differentiated mast cells. It also affected human mast cell and basophil activation. Inhibition required a few hours to establish and lasted for a few hours only. It probably involved cell-bacteria contact and soluble bacterial constituents. Similar observations were reported with other bacteria. Nonpathogenic E. coli strains were found to inhibit mouse and human mast cell activation in vitro. How commensal bacteria could reach mast cells in vivo is intriguing. Mast cells are present in every tissue but commensals mostly reside in the gut. Bacteria can be taken up by dendritic or M cells and cross the gut epithelium, but in low numbers. Bacteria are transiently found in blood, especially after meals. LPS circulates in plasma, and it favors insulin resistance and obesity by inducing pro-inflammatory cytokines.

Soluble bacterial products interact with mast cells via TLRs. Thus, when engaged respectively by proteoglycans or LPS, TLR2 and TLR4 activate different sets of genes and induce different sets of cytokines in mast cells. The inventors failed to identify receptors involved in inhibition of mast cell activation by L. casei. That inhibition occurred in MyD88^{-/-} cells excludes TLR2, 4, 5, 7 and 9, and inhibition did occur in TLR2/4^{-/-} cells. The inventors did not study TRIF^{-/-} cells, but inhibition was unaffected in cells lacking TLR4 or TLR3 which signal through TRIF. Inhibition was unaffected either in cells lacking NOD2 which binds muramyl dipeptide and promotes apoptosis, FcγRIIB which binds viral and fungal molecules and negatively regulates cell activation, FcγRIIIA which binds E. coli and can generate inhibitory signals, FcRγ which mediates FcR signaling or DAP12 which mediates the inhibition of FcR and TLR signaling by TREM-2. The inventors therefore could not identify any specific inhibitory pathway. The inventors' analysis of FcεRI-dependent signals, however, indicates that L. casei has multiple targets. Both upstream events, such as Syk, LAT, PLC-γ and Gab2 phosphorylation, and downstream events, such as Erk1/2, JNK, p38, I-κB and NF-κB phosphorylation, were affected. Alterations of downstream signals could result from alterations of upstream signals. Ionomycin- and thapsigargin-induced signals, which bypass upstream signals, were however inhibited, indicating that downstream signals were targeted by bacteria. A major consequence was an abrogation of Ca²⁺ responses. Noticeably, Akt phosphorylation, which has anti-apoptotic effects was the only enhanced signal. In accordance with these results. L. casei was reported to down regulate the expression of genes encoding pro-inflammatory cytokines by inhibiting the NF-κB pathway through stabilization of I-κB in Shigella-infected intestinal epithelial cells. Cells other than mast cells can indeed be affected by L. casei, but not necessarily through identical mechanisms. The anti-inflammatory effects of L. casei-treated dendritic cells in trinitrobenzenesulfonic acid-induced colitis required MyD88, TLR2 and NOD2.

The inventors have found that mast cells play a pivotal role in the regulation of inflammation by the bacterial flora. Mast cells are the main effectors of IgE-dependent acute reactions, such as anaphylaxis, but not of chronic allergic reactions, such as asthma, in which cellular infiltrates are prominent. They, however, are essential for the initiation of not only IgE-, but also IgG-dependent chronic inflammation. The engagement of mast cell FcRs indeed controls the release of vasoactive amines and proteases, the synthesis of lipid-derived mediators, the secretion of chemokines and cytokines. These mediators concur to attract and activate inflammatory cells. Pathogenic bacteria can activate mast cells and amplify their pro-inflammatory effects. The inventors show here that a commensal bacteria markedly dampens mast cell responses. Regulation of inflammation by mast cells is therefore controlled both by FcRs and by microorganisms, and in both cases, positively and negatively. This role played by mast cells in the effector phase of immune responses is reminiscent of the role played by dendritic cells in the afferent phase of immune responses. Dendritic cells are activated by pathogen products, leading to their maturation which is required for antigen presentation to T cells, and commensals modulate their differentiation. Like mast cells, they express activating FcRs for IgG and IgE and inhibitory FcRs, the engagement of which by immune complexes also alter their activation and differentiation. The role assigned to microorganisms in the hygiene hypothesis has been based mostly on their effects on the control of Th1/Th2 polarization by dendritic cells. The inventors show that their effects on the control of inflammation by mast cells explain the parallel increased incidence of Th1- and Th2-type inflammatory immune diseases.

A first aspect of the present invention is hence the use of a L. casei strain, for the preparation of a composition for inhibiting mast cell activation, in particular for preventing, alleviating or treating a disease or a disorder involving or mediated by mast cell activation.

Depending on the clinical context and on the route of administration, the compositions prepared according to the invention can inhibit IgE- and/or IgG-induced mast cell activation. They can hence be used to prevent, alleviate and/or treat any inflammatory manifestation implying mast cell activation by antibodies in the presence of antigens.

In particular, when the IgE-induced activation is inhibited, these compositions can be used for preventing, alleviating and/or treating an allergy or allergic manifestations. The allergies considered herein are caused by IgE antibodies which bind to mast cells and, when recognizing specific antigens, trigger their activation. Importantly, these compositions can be used to prevent, treat or alleviate allergic manifestations (e.g., asthma, rhinitis or hay fever, allergic eczema, anaphylactic shock etc.), even in subjects who have already been sensitized to an antigen, and who have already been diagnosed as allergic to this antigen. For example, a person who has suffered for many years from hay fever can prevent the reappearance of the symptoms by taking compositions prepared according to the invention. A huge number of antigens can cause allergies, which can manifest themselves in a great variety of clinical symptoms. Non-limitative examples of antigens frequently at the origin of allergies are environmental allergens such as mite (e.g., Der p 2), cockroach antigens, birch pollen (e.g. Bet V 1), grass pollen, animal hair dander antigens (e.g., Cat: Fel d 1), bee venom (e.g., phospholipase), or food allergens such as milks (especially cow milk), peanut, shrimp, soya, eggs, cereal products, fruits, etc. Depending on the context and the individual, the clinical symptoms can be local (which is the case, for example, in allergic rhinitis, conjunctivitis or otitis), regional (e.g., asthma, dermatitis, gastroenterological problems and Quincke's oedema), or general (e.g., anaphylactic shock). Some pathologies are sometimes abusively defined as allergies, although they do not depend on the above-recalled mechanism. This is the case, for example, of delayed-type hypersensitivity reactions.

When IgG-induced mast cell activation is inhibited by a composition obtained according to the present invention, this composition can advantageously be used for preventing, alleviating or treating an autoimmune disease, such as for example rheumatoid arthritis, encephalomyelitis, multiple sclerosis, bullous pemphigoid, acute disseminated encephalomyelitis (ADEM), ankylosing spondylitis, antiphospholipid antibody syndrome (APS), autoimmune hepatitis, autoimmune oophoritis, celiac disease, Crohn's disease, gestational pemphigoid, Goodpasture's syndrome, Graves' disease, Guillain-Barré syndrome (GBS, also called acute inflammatory demyelinating polyneuropathy, acute idiopathic polyradiculoneuritis, acute idiopathic polyneuritis and Landry's ascending paralysis), Hashimoto's disease, idiopathic thrombocytopenic purpura, Kawasaki's disease, lupus erythematosus, myasthenia gravis, opsoclonus myoclonus syndrome (OMS), optic neuritis, Ord's thyroiditis, pemphigus, Reiter's syndrome, Sjögren's syndrome, Takayasu's arteritis, temporal arteritis (also known as “giant cell arteritis”) and Wegener's granulomatosis. Importantly, and since the compositions according to the invention have an effect on the effector phase of these diseases, patients already suffering from auto-immune diseases can benefit from these composition. Any other auto-immune disease which depends on mast cell activation by antibodies can also be prevented or treated by a composition obtained according to the invention.

In a preferred embodiment, the L. casei strain used according to the present invention is a L. casei ssp. paracasei, for example the strain deposited at the CNCM (Collection Nationale de Culture de Microorganismes, 25 rue du Docteur Roux, Paris) under the number I-1518 on Dec. 30, 1994.

According to a particular embodiment of the present invention, the composition prepared with a L. casei strain is a food supplement and/or a functional food. In the present text, a “food supplement” designates a product made from compounds usually used in foodstuffs, but which is in the form of tablets, powder, capsules, potion or any other form usually not associated with aliments, and which has beneficial effects for one's health. A “functional food” is an aliment which also has beneficial effects for one's health. In particular, food supplements and functional food can have a physiological effect—protective or curative—against a disease, for example against a chronic disease.

Compositions obtained according to the present invention can also comprise at least one other bacterial strain selected from the genera Lactobacillus, Lactococcus and Streptococcus, for example at least one bacterial strain selected in the group consisting of Streptococcus thermophilus and Lactobacillus bulgaricus.

According to another embodiment, the composition prepared according to the present invention is a medicinal product.

In accordance with the invention, the bacteria, especially the L. casei bacteria, can be used in the form of whole bacteria which may be living or not. For example, whole irradiated L. casei can be used. Alternatively, bacteria can be used in the form of a bacterial lysate or in the form of bacterial fractions; the bacterial fractions suitable for this use can be chosen, for example, by testing their properties of inhibiting IgE-induced mast cell activation, for example by performing one of the assays disclosed in the experimental part below. Bacterial fractions are especially preferred, for example, in formulations targeting the mucous membrane of nose and sinus, the lung, the peritoneum . . . .

In a preferred embodiment, the compositions obtained according to the present invention are formulated to enable a direct contact between mast cells and bacteria, bacterial lysate and/or the bacterial fraction (possibly partially degraded).

The effects of L. casei and, to a lesser extent, of B. breve on the inhibition of mast cell activation, might also be observed with other bacterial strains. Libraries of bacterial strains, especially of already known probiotics, should be screened for identifying further strains able to inhibit mast cell activation.

The present invention hence also pertains to screening processes for identifying bacterial strains or bacterial fractions which can be used for preparing compositions for inhibiting mast cell activation, particularly activation by antibodies, especially for preventing, alleviating or treating a disease selected amongst allergies and autoimmune diseases.

According to a first variant of the processes (first process) according to the invention, said process comprises the following steps:

a) administering an amount of bacterial composition to a normal mouse by parenteral route,

b) subsequently injecting intravenously the serum from an autoimmune K/B×N mouse; and

c) determining the severity of arthritis in said normal mouse, optionally by analyzing the score of arthritis by clinical examination. The L. casei strain CNCM I-1518 may be used as a positive control in this method. Subcellular components or fractions of bacteria or inactivated bacteria may also be screened using this method.

According to a second variant of the processes (second process) according to the invention, said process comprises the following steps:

a) administering orally or parenterally a bacterial composition (whole cells, live or killed, or a fraction thereof) to normal mice,

b) subsequently injecting intravenously said normal mice with purified IgE or serum containing IgE antibodies specific for an allergen,

c) challenging intravenously said IgE-injected mice, optionally one or more days later, with said allergen,

e) determining the intensity anaphylactic shock induced by said challenge, optionally by measuring a decrease in body temperature,

wherein a reduced anaphylactic shock compared to control mice not administered said bacterial composition is indicative of a bacterial composition which treats or prevents a mast cell dependant allergic process. L. casei CNCM I-1518 may be employed as a positive control in this method.

The present invention also pertains to a process for identifying or screening a molecule which decreases the anaphylactic reaction in vivo comprising:

a) pre-incubating mast cells with a molecule of interest,

b) incubating the mast cells with purified IgE specific for an allergen,

c) injecting normal mice with said mast cells and with the allergen, and

d) determining the degree of anaphylactic reaction in said mice, optionally by monitoring and/or analyzing said mice for changes in body temperature or for mortality;

wherein a reduced amount of anaphylactic reaction in the treated mice compared to control mice administered mast cells not treated with said molecule of interest indicative of molecule which decreases the anaphylactic reaction in vivo. This process may be used to screen various biological, chemical or pharmaceutical molecules, including components of Lactobacillus including cytoplasmic molecules, peptidoglycan, or components of the Lactobacillus membrane.

Another aspect of the present invention is a process of testing new molecules by using an adoptive anaphylaxis reaction comprising:

a) sensitizing in vitro mast cells by contacting them with IgE specific for an allergen of interest,

b) injecting the sensitized mast cells to normal mice, subsequently

c) injecting into said mice the allergen of interest,

d) administering the molecule to be tested to said mice, and

e) monitoring the anaphylactic shock optionally by measuring the body temperature of said mice injected with the allergen of interest. The allergen of interest is as described above, and the molecule to be tested can be administered by any route.

The experimental part below and the figures will further illustrate the present invention, without limiting its scope.

### EXAMPLES

### Experimental Procedures

Mast cells. BMMC were obtained by culturing femoral bone marrow from C57BL/6 (Charles River, L'Arbresle, France), TLR2/4^{-/-} (Ramphal et al., 2005), TLR3^{-/-} (Alexopoulou et al., 2001), MyD88^{-/-} (Kawai et al., 1999) (a gift of Dr. M. Chignard, Institut Pasteur, Paris, France), NOD2^{-/-} (Barreau et al., 2007) (a gift of Dr. I. Boneca, Institut Pasteur, Paris, France), FcRγ/DAP12^{-/-} (a gift of Dr. E. Vivier, CIML, Luminy, France) and from corresponding littermate controls mice in IL-3-containing medium as described (Malbec et al., 2007). PCMC were obtained by culturing peritoneal cells from C57BL/6 mice in SCF-containing medium as described (Malbec et al., 2007).

Bacteria. Lactobacillus casei (Strain DN-114 001, CNCM I-1518), Bifidobacterium breve (Strain BBC50, CNCM I-2219) and Streptococcus thermophilus (Strain 001 236, deposited at the CNCM under the number 1-2273 on Jan. 24, 2002) were from the Danone library. Live L. casei was cultured in Mann-Rogosa-Sharpe (MRS) broth (Becton Dickinson, Mountain view, Calif.) at 37° C. without agitation and in absence of oxygen. Bacteria were γ-irradiated at 10K Gy. FITC-labeled γ-irradiated L. casei was prepared by incubating γ-irradiated bacteria at 1×10⁹/ml with 0.5 mg/ml fluorescein isothiocyanate (FITC) (Sigma, Saint Louis, Mo.) in 50 mM NaHCO₃, 100 mM NaCl, pH 9.0 for 20 min at room temperature in the dark. Bacteria were washed and resuspended in PBS.

Mast cell stimulation. Stimulation by bacteria: Unless otherwise specified, BMMC were incubated overnight at 37° C. with γ-irradiated (1000 bacteria/cell) or live (100 bacteria/cell) L. casei, B. breve or S. thermophilus. Stimulation by IgE and antigen: BMMC or PCMC were sensitized with 1 μg/ml mAb IgE anti-DNP 2682-I (Liu et al., 1980) for 1 h, washed, and challenged with DNP-BSA. Stimulation by IgG immune complexes: PCMC were challenged with preformed immune complexes for 20 min at 37° C. Immune complexed were made by incubating 100 μg/ml Glucose phosphate isomerase (GPI) (Sigma) with the indicated dilutions of serum anti-GPI for 15 min at 37° C. immediately before use. Nonspecific stimulation: BMMC were treated with either phorbol myristic acetate (PMA) (Sigma) plus ionomycin (Sigma), ionomycin, or thapsigargin (Sigma) at the indicated concentration and for the indicated periods of time.

TNF-α titration. Mast cells stimulated for 3 h at 37° C. were centrifuged, and TNF-α contained in cell supernatant was titrated by a cytotoxicity assay on L929 cells as described (Latour et al., 1992).

β-hexosaminidase measurement. Mast cells stimulated for 20 min at 37° C. were centrifuged, and β-hexosaminidase contained in cell supernatant was quantitated using an enzymatic assay as described (Malbec et al., 2007).

Interleukine measurement. Twenty interleukins, chemokines or growth factors were also measured in 3-h supernatants using the mouse cytokine twenty-plex kit (Biosource, Nivelles, Belgium) and a Luminex 100IS System (Luminex, Austin, Tex.).

Calcium mobilization. Mast cells were loaded with 0.5 μM Fluo-3-AM (Invitrogen, Carlsbad, Calif.) for 1 h at room temperature, washed and analyzed by flow cytometry (Becton Dickinson) before and after stimulation with the indicated reagents.

Western blot analysis. Mast cells sensitized with IgE anti-DNP 26821 and challenged with 10 ng/ml DNP-BSA for 0, 3, 10 and 30 min were centrifuged. Cell pellets were resuspended in Tris SDS, boiled for 5 min, fractionated by SDS-PAGE and transferred onto nitrocellulose membranes. After blocking with 5% skimmed milk (Régilait, Saint-Martin-Belle-Roche, France), membranes were incubated with rabbit anti-phospho-Akt (Cell Signaling Technology, Beverly, Mass.), rabbit anti-phospho-Gab2 (Cell Signaling), rabbit anti-phospho-1-κBα (Cell Signaling), rabbit anti-phospho-LAT (Upstate Biotechnology, Lake Placid, N.Y.), rabbit anti-phospho-NF-κB p65 (Cell Signaling), rabbit anti-phospho-p38 MAPK (Cell Signaling), rabbit anti-phospho-p44/42 MAPK (Cell Signaling), rabbit anti-phospho-PLCγ1 (Santa Cruz Biotechnology, Santa Cruz, Calif.), rabbit anti-phospho-JNK (Cell Signaling), rabbit anti-phospho-Syk (Cell Signaling), rabbit anti-Akt (Cell Signaling), rabbit anti-Gab2 (Upstate), rabbit anti-I-κBα (Cell Signaling), rabbit anti-LAT (Upstate), rabbit anti-NF-κB p65 (Cell Signaling), rabbit anti-p38 MAPK (Cell Signaling), rabbit anti-p44/42 MAPK (Cell Signaling), mouse anti-PLCγ1 (Santa Cruz), rabbit anti-JNK (Cell Signaling), rabbit anti-Syk (Santa Cruz Biotechnology) or goat anti-actin (Santa Cruz). Membranes were probed with either goat anti-rabbit IgG-HRP (Santa Cruz), rabbit anti-goat IgG-HRP (Santa Cruz) or goat anti-mouse IgG-HRP (Santa Cruz). Labeled Abs were detected using an ECL kit (Amersham Biosciences, Buckinghamshire, United Kingdom).

Mast cell viability. Mast cells were incubated overnight with PBS or γ-irradiated L. casei at a ratio of 1,000 bacteria/cell or 0.25 μM staurosporin (Sigma). Cells were washed, stained with 0.5 μg/ml propidium iodide (Sigma) and annexin V-allophycocyanin (Becton Dickinson), and analysed by flow cytometry (Becton Dickinson).

Phagocytosis assay. BMMC or THP-1 cells were mixed with live L. casei, spinoculated at 600 g for 10 min at 4° C., incubated for 35 min at 4° C. or 37° C., washed, treated or not with 1 mg/ml gentamycin (Euromedex, Souffelweyersheim, France) and lysed with 0.1% TX-100 (Sigma). Lysates were plated onto MRS agar (Becton Dickinson) and incubated at 37° C. for 48 h.

Flow cytometry analysis of cell-bound extracellular bacteria. Mast cells were incubated for 45 minutes at 0° C. or 37° C. with FITC-labeled γ-irradiated L. casei at a ratio of 1,000 bacteria/cell. Cells were washed, treated or not with 250 μg/ml trypan blue (Sigma) to quench the fluorescence of extracellular bacteria, and analysed by flow cytometry (Becton Dickinson).

Confocal video microscopy. Mast cells were stained with 5 μM SNARF (Invitrogen) for 10 min at 37° C., washed, and incubated with FITC labeled γ-irradiated L. casei at a ratio of 1.00 bacteria/cell overnight at 37° C. Cells were then washed, resuspended in phenol red-free RPMI 1640 (Invitrogen), and directly analysed by confocal microscopy (Zeiss, Jena, Germany).

Passive Systemic Anaphylaxis. 6-8 week old C57BL/6J mice (Charles River) were given live L. casei either intraperitoneally (5×10⁷ in 200/L PBS) or in drinking water (1×10⁸/mL) for the indicated time. Mice were injected intravenously with 50 μg mAb IgE anti-DNP SPE-7 (mAb SPE-7, Sigma) on day −1 followed by 500 μg DNP₁₅-BSA on day 0, or with 200 g mAb IgE anti-ovalbumin 2C6 (mAb 2C6) (Hamada et al., 2003) on day −1 followed by 1 mg ovalbumin on day 0. Rectal temperature was assessed using a Precision digital thermometer 4600 (YSI, Dayton, Ohio, USA).

K/B×N arthritis. Transgenic KRN mice on the C57BL/6 background were crossed to NOD mice (Charles River) to generate K/B×N mice. K/B×N serum collected over several weeks was pooled and kept frozen. 6-8 week old C57BL/6J mice (Charles River) were given live L. casei either intraperitoneally (5×10⁷ in 200 μL PBS) or in drinking water (1×10⁸/mL) and by gavage (2×10⁹ in 200 μL PBS) for the indicated time period. One intravenous injection of 50/L K/B×N serum consistently induced arthritis in wild type mice. Arthritis was scored by clinical examination as described (Bruhns et al., 2003) (index of all 4 paws were added: 0 (unaffected), 1 (swelling of one joint), 2 (swelling of more than one joint) and 3 (severe swelling of the entire paw)). Ankle joints were fixed, decalcified in Formal-4 (Decal Corporation, Tallman, N.Y.) for 7 days, embedded in paraffin and stained with hematoxylin/eosin.

Human basophil activation. Red cell-depleted blood cells from normal donors (from the Centre Cabanel of the Etablissement Français du Sang) were resuspended at 10⁶ cells/mL in complete culture medium and incubated overnight with PBS or with the indicated numbers of γ-irradiated L. casei. Aliquots of 1×10⁶ cells were incubated for 30 min at 37° C., with 5 ug/mL rabbit anti-human IgE F(ab′)₂ fragments or with medium alone. F(ab′)₂ was obtained by digesting rabbit anti human IgE antibodies (DAKO, Oxford, UK) for 48 h at 37° C. with 2% pepsine in acetate buffer pH 3.0. Cells were labeled with anti allophycocyanine-labeled anti-FcεRI (eBioscience, San Diego, USA) and phycoerytrine-labeled CD203c antibodies (Beckman Coulter, Marseille, France). Cell fluorescence was monitored with FACScalibur (Becton Dickinson Bioscience, Le Pont de Claix, France).

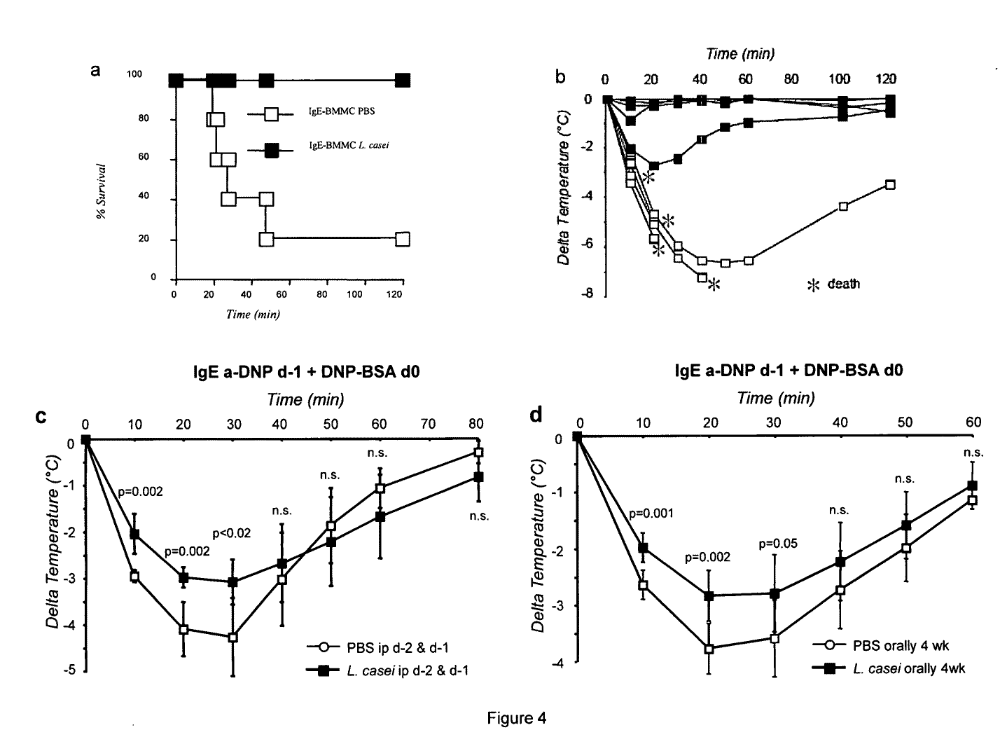

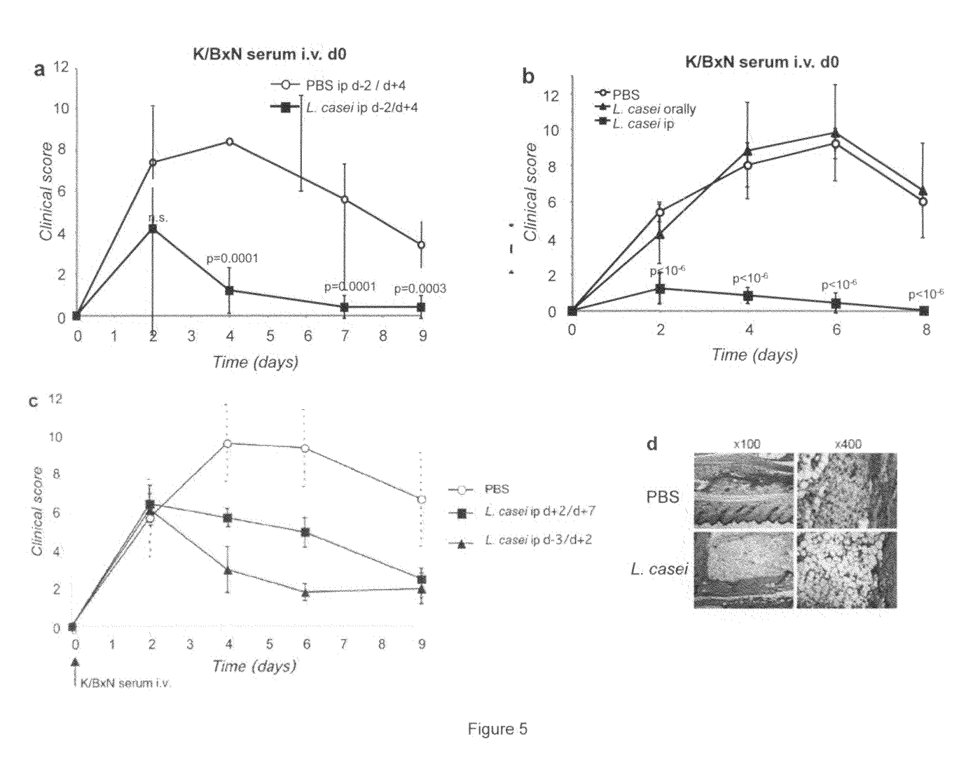

Statistical analyses. Data shown in FIGS. 4 and 5 are the means±s.e.m. Data were analyzed for statistical significance using Student's t-test. p values higher than 0.05 were considered as not statistically significant.

### Example 1

### L. Casei Inhibits Mast Cell Activation

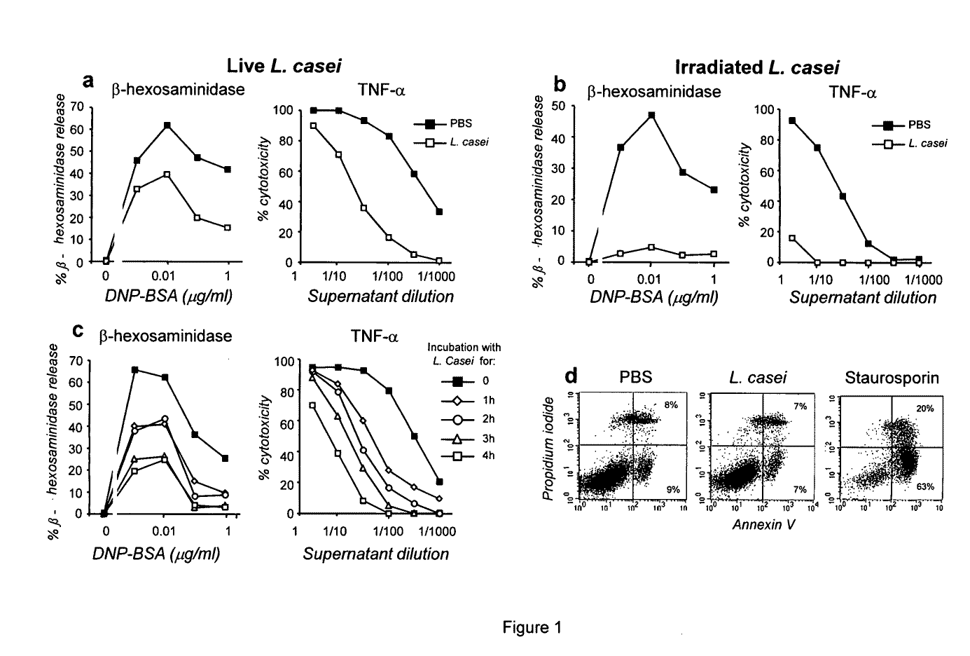

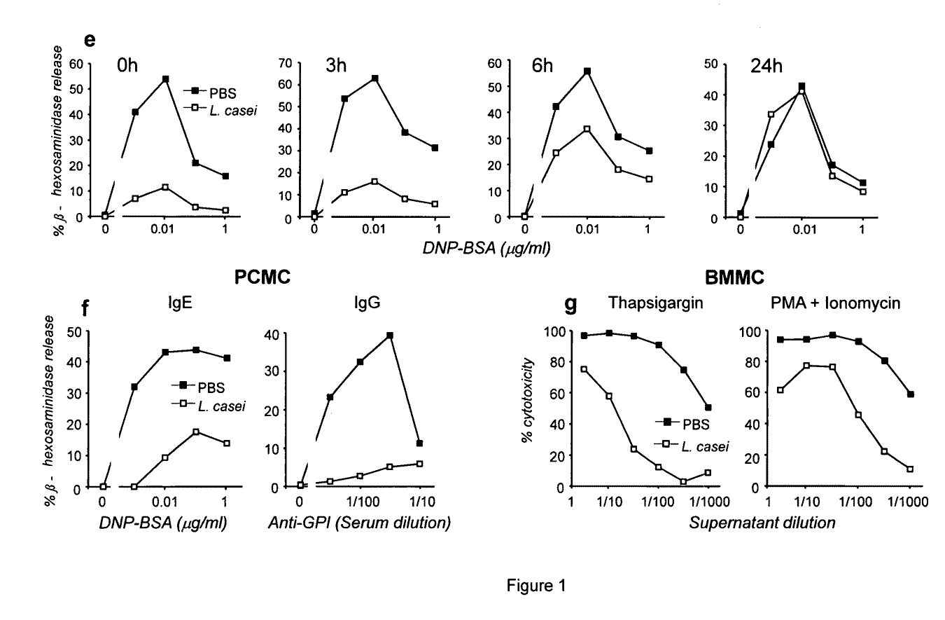

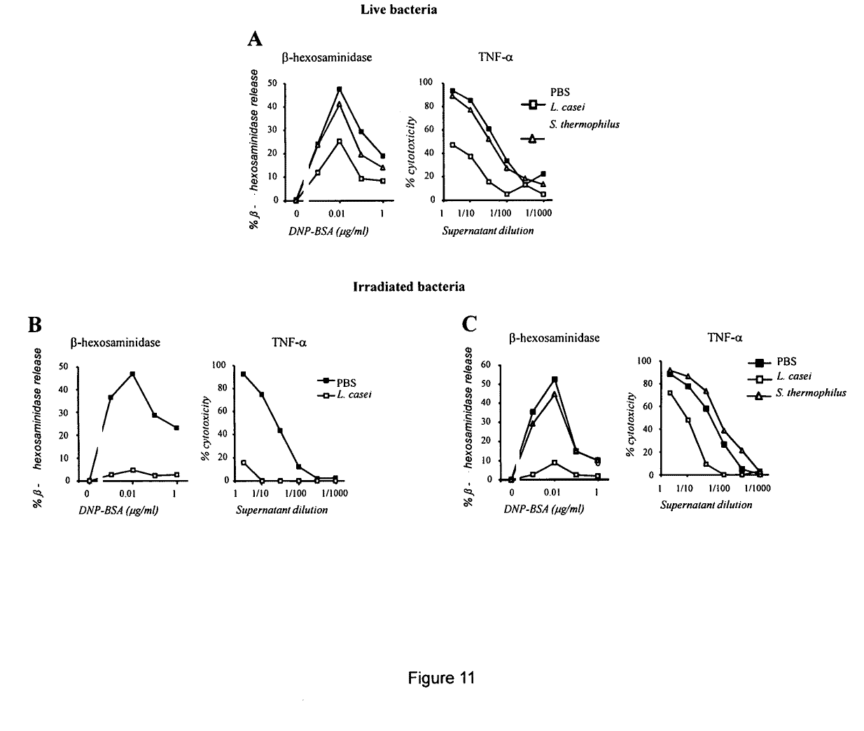

When sensitized with IgE antibodies and challenged with antigen, Bone Marrow-derived Mast Cells (BMMC) release granular mediators such as β-hexosaminidase and secrete cytokines such as TNFα. When incubated overnight with L. casei before sensitization with IgE, BMMC released lower percentages of the granular enzyme β-hexosaminidase (2-fold more) and secreted less TNF-A upon antigen challenge (FIG. 1a and FIG. 11A).

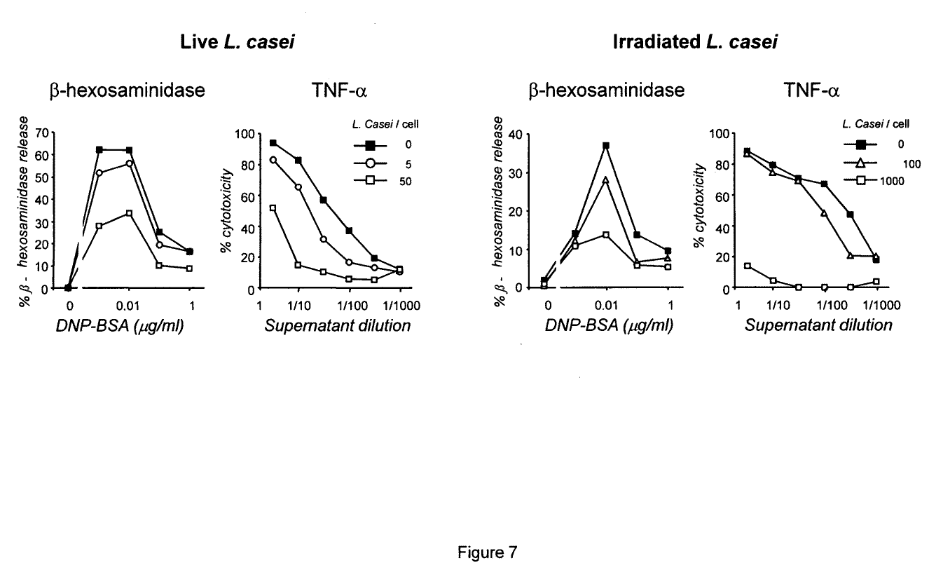

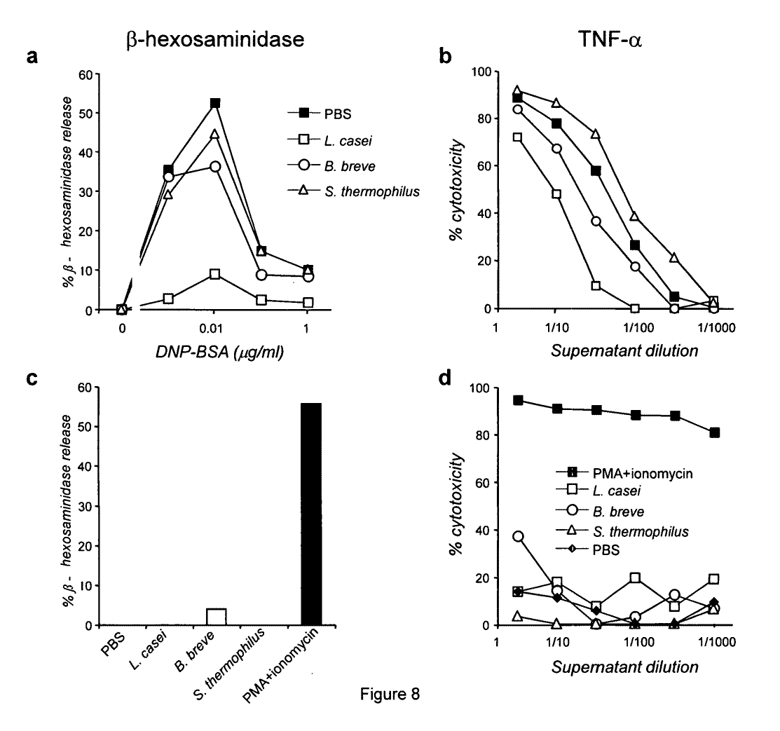

L. casei secretes lactic acid and other metabolites which could possibly account for the observed inhibition. An overnight incubation of BMMC with high concentrations of lactic acid (up to 20 mg/ml), however, did not affect IgE-induced secretory responses (not shown). To determine whether bacteria themselves could inhibit mast cell activation, BMMC were incubated overnight with γ-irradiated L. casei (which do not secrete metabolites) before they were sensitized with IgE. Irradiated L. casei also inhibited IgE-induced β-hexosaminidase release and TNF-α secretion (FIG. 1b and FIGS. 11B & 11C). Inhibition, however, required more γ-irradiated than live bacteria (FIG. 7). Two other γ-irradiated commensal bacteria, Bifidobacreium breve or Streptococcus thermophilus, inhibited neither β-hexosaminidase release nor TNF-α secretion (FIG. 8a and FIG. 11). None of the three bacteria induced BMMC to release β-hexosaminidase or to secrete TNF-A (FIG. 8b).

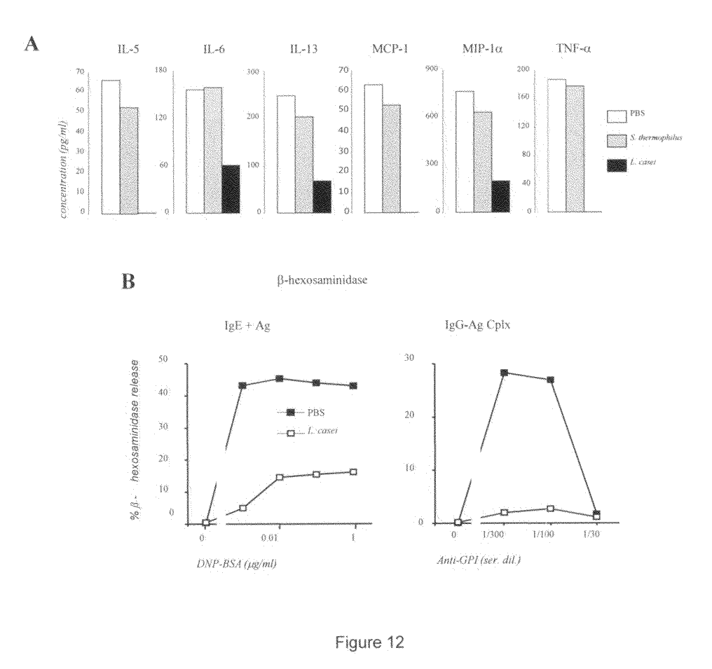

When challenged for 3 h with antigen, BMMC sensitized with IgE antibodies secreted TNF-α, but also IL-5, IL-6, IL-13, MCP-1 and MIP-1α, but not IL-1α, IL-1β, IL-2, IL-4, IL-10, IL-12, IL-17, IP-10, KC, MIG, VEGF, FGF, GM-CSF or IFN-γ, as assessed by the Luminex Technology (not shown). The secretion of all antigen-induced cytokines and chemokines was inhibited when BMMC were incubated overnight with L. casei, but not with S. thermophilus, before sensitization with IgE antibodies. The secretion of IL-5, MCP-1 and TNF-α was below the detection limits of Luminex and that of IL-6, IL-13 and MIP-1α was markedly reduced (FIG. 12A).

BMMC are immature mast cells. The inventors recently described a novel model of mature serosal-type mast cells (Malbec et al., 2007). Unlike BMMC, but like normal peritoneal mast cells, these Peritoneal Cell-Derived Mast Cells (PCMC) degranulate in response not only to IgE and antigen, but also to IgG immune complexes. These Peritoneal Cell-derived Mast Cells (PCMC) indeed released β-hexosaminidase when challenged with preformed immune complexes made with GPI and the same K/B×N serum which was used to induce arthritis. IgE- and IgG-induced β-hexosaminidase release were both inhibited when PCMC were incubated overnight with live L. casei before sensitization with IgE or before challenge with immune complexes (FIG. 12B). An overnight incubation of PCMC with γ-irradiated L. casei similarly inhibited both IgE- and IgG-induced β-hexosaminidase release (FIG. 1f). Surprisingly, L. casei also inhibited TNF-A secretion by BMMC in response to thapsigargin or PMA and ionomycin (FIG. 1f).

These results indicate that an incubation of mast cells, either immature or mature, with the commensal bacteria L. casei for a few hours profoundly but reversibly inhibits secretory responses subsequently induced by immunological and nonimmunological stimuli. L. casei, either live or irradiated, can therefore inhibit all in vitro secretory responses of mast cells induced by the same IgE or IgG antibodies that induced systemic anaphylaxis or arthritis in vivo.

### Example 2

### Inhibition Requires a Contact Between Cells and Bacteria

Inhibition of mast cell activation depended on the duration of incubation of cells with L. casei. A 1-h incubation with γ-irradiated bacteria was sufficient to induce a detectable inhibition, and inhibition further increased with the duration of incubation (FIG. 1c).

Inhibition could not be accounted for by cell death as similarly small percentages of BMMC were labeled with annexin V and/or propidium iodide, following an overnight incubation with PBS or with γ-irradiated L. casei (FIG. 1d). In accordance with this observation, inhibition was reversible. β-hexosaminidase release, which was virtually abolished when mast cells were sensitized with IgE and challenged with antigen immediately after the overnight incubation with γ-irradiated bacteria, was partially restored when sensitization and challenge were delayed for 6 h. It was back to normal after 24 h (FIG. 1e).

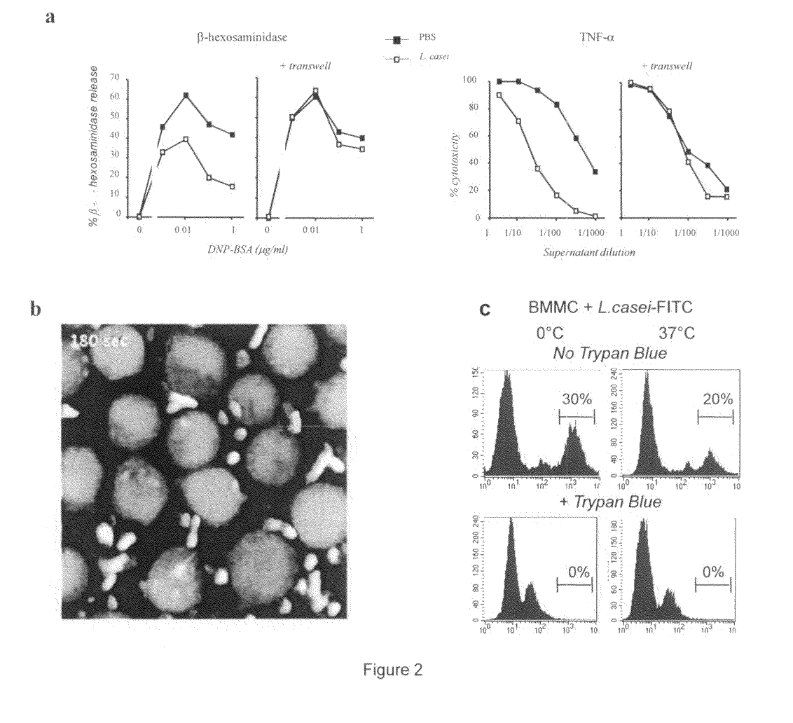

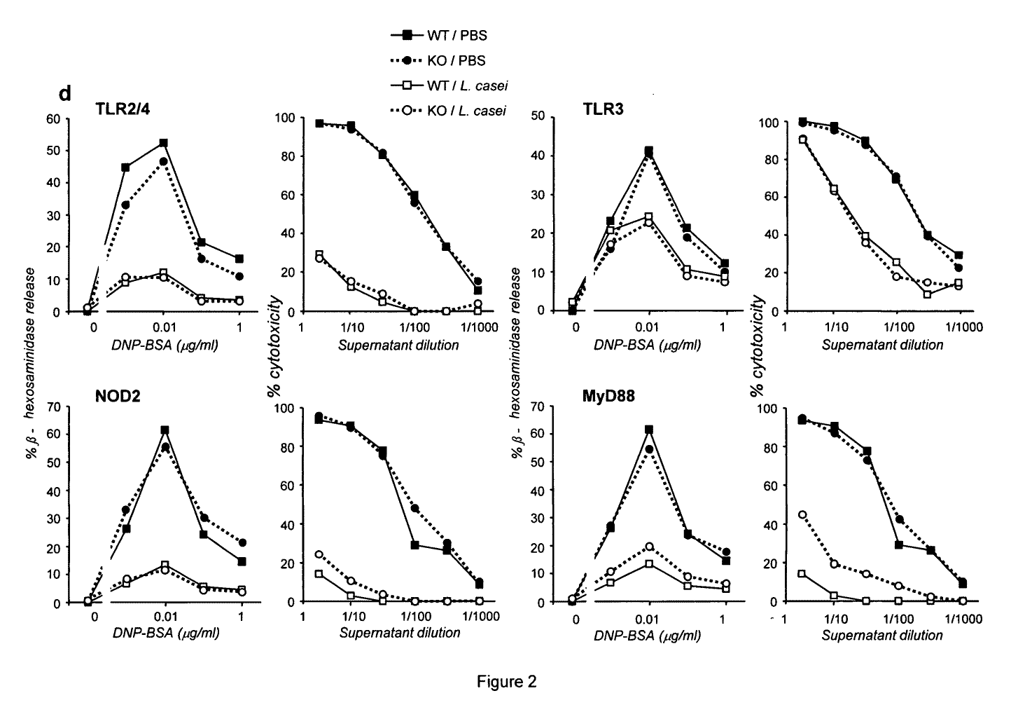

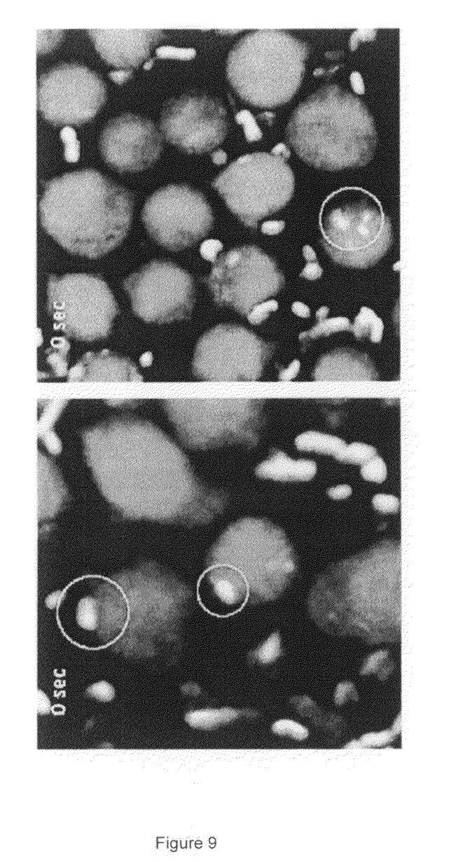

Inhibition of mediator release and cytokine secretion observed following an overnight incubation of mast cells with L. casei was abrogated when cells were separated from bacteria by a porous membrane (pore size 0.4 μm) during incubation (FIG. XX). Supporting this observation, FITC-labeled L. casei could be observed attached to and possibly inside mast cells, when examined by real-time confocal imaging after an overnight incubation (FIG. 2b and FIG. 9).

Cell-associated FITC-labeled γ-irradiated L. casei were also seen by flow cytometry following a 45-min incubation of BMMC with bacteria at 0° C. or at 37° C. (FIG. 2c). Cell-bound fluorescence was similarly quenched by trypan blue, whether cells were incubated with bacteria at 0° C. or at 37° C., suggesting that bacteria were not internalized. No detectable phagocytosis of live L. casei by BMMC was observed either in a gentamycin protection assay (not shown).

These data altogether indicate that inhibition of mast cell activation by L. casei requires a contact between cells and bacteria.

### Example 3

### L. Casei Interacts with Mast Cells Via an Unknown Receptor

In an attempt to identify a receptor possibly involved in inhibition of mast cell activation by L. casei, BMMC from various knock-out mice and their littermate controls were studied. These included mice lacking receptors or signaling molecules involved in the recognition of microbial products. L. casei-induced inhibition was unaffected by the deletion of TLR2+4, TLR3, NOD2, or MyD88 (FIG. 2d). It was unaffected either by the deletion of FcγIIB, FcγRIIIA, FcRγ or DAP12 (not shown). L. casei therefore inhibits mast cell activation by interacting, directly and/or via the release of soluble material, with an unknown receptor.

### Example 4

### L. Casei Inhibits Major Intracellular Signaling Pathways

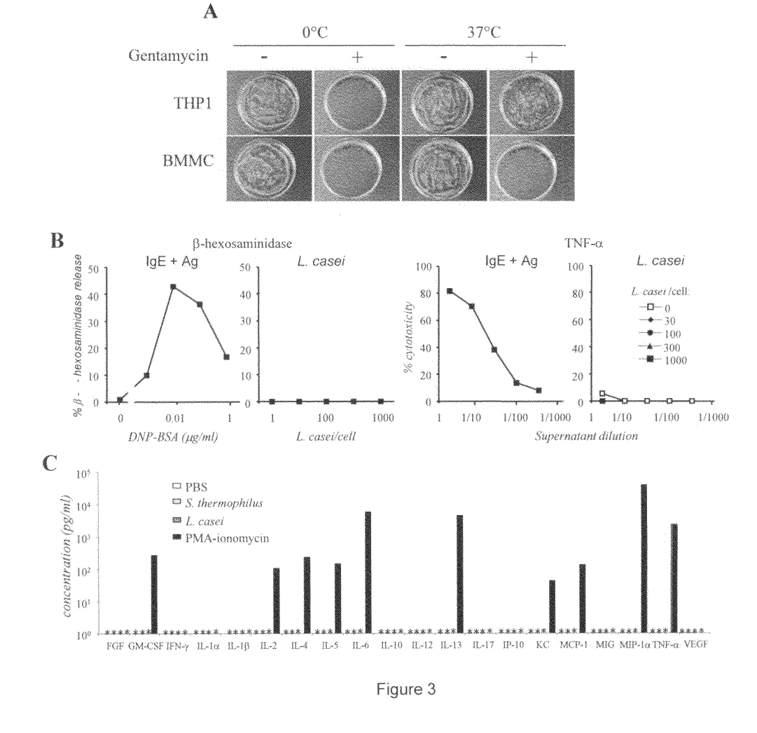

The possibility that L. casei was phagocytosed by mast cells was examined by a gentamycin-protection assay. BMMC or, as a positive control, the macrophage-like cells THP1 were incubated with L. casei at 0° C., enabling bacteria to bind onto cells, but not to be phagocytosed, or at 37° C., enabling both binding and phagocytosis. Cells were subsequently treated with gentamycin, which can kill extracellular, but not intracellular bacteria, and phagocytosis was assessed by bacterial growth. No detectable phagocytosis of L. casei by BMMC was observed under these conditions (FIG. 3A).

Inhibition could possibly result from “desensitization” of mast cells, as a consequence of a previous activation by L. casei. The inventors therefore investigated whether bacteria could induce mediator release or cytokine secretion following a 20 min- or a 3 h-incubation with mast cells. IgE+antigen or PMA+ionomycin, and S. thermophilus were used as positive and negative controls, respectively. L. casei induced neither β-hexosaminidase release nor TNF-α secretion (FIG. 3B). L. casei also failed to induce the secretion of any cytokine, chemokine or growth factor (FIG. 3C). Noticeably, L. casei induced no detectable secretion of IL-10.

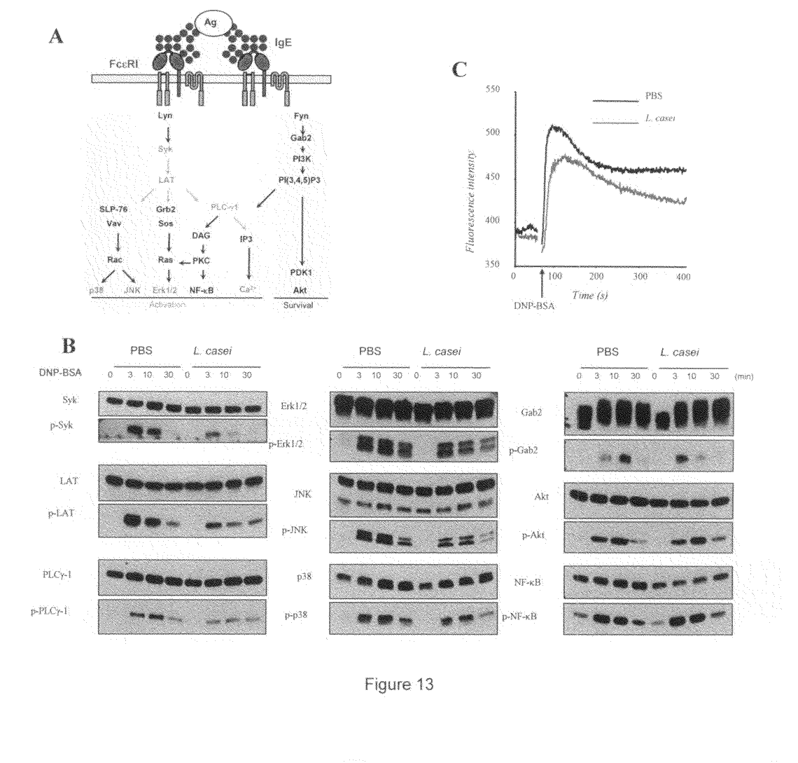

As L. casei did not decrease FcεRI expression (not shown), the inventors examined FcεRI-dependent intracellular signaling in BMMC incubated overnight with bacteria before sensitization with IgE and challenged with antigen for various periods of time They found that L. casei affected both proximal and distal FcεRI signaling (FIG. 13A). The Lyn-initiated pathway was dampened as shown by a reduced phosphorylation of Syk, LAT and PLC-γ, and the three MAP kinases Erk1/2, JNK and p38 were more transiently phosphorylated. NF-κB phosphorylation was however not detectably affected. Likewise, neither proximal (Gab2 phosphorylation) nor distal (Akt phosphorylation) events were impaired in the Fyn-initiated pathway (FIG. 13B). Ultimately, IgE-induced increase of intracellular Ca²⁺ concentration was reduced in BMMC exposed to L. casei (FIG. 13C).

These results altogether indicate that L. casei seems unable to activate mast cells, as judged by secretion and/or phagocytosis, and seems not to use microbial receptors that mediate cell activation by bacteria. L. casei rather inhibits FcεRI signaling by affecting selectively the Lyn/SyK/LAT pathway.

### Example 5

### L. Casei Inhibits IgE- and IgG-Dependent Inflammation In Vivo

The inventors investigated whether L. casei could have in vivo effects in murine models of inflammation which depend on mast cells. In a first model of adoptive anaphylaxis, mice were injected intravenously with BMMC sensitized with IgE. An intravenous injection of antigen 15 min later induced a severe shock leading to the death of 80% mice (FIG. 4a) and a major drop in body temperature (FIG. 4b). All mice injected with BMMC incubated overnight with L. casei before IgE sensitization survived (FIG. 4a) and they displayed no or a mild shock (FIG. 4b).

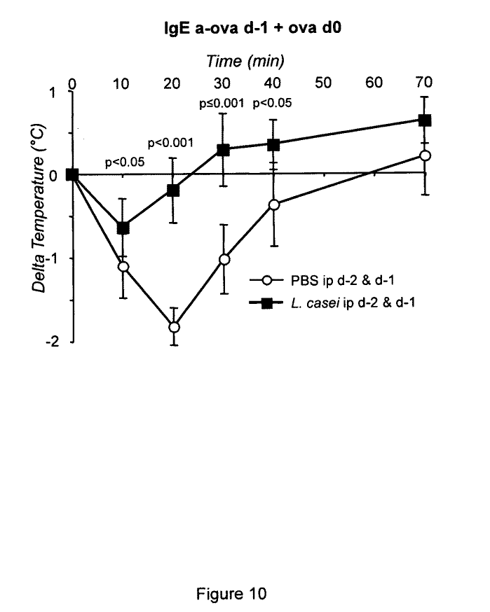

In a second model of passive systemic anaphylaxis, mice were injected intravenously with IgE one day before antigen challenge at day 0. An intraperitoneal injection of L. casei at days −2 and −1 significantly reduced the drop in body temperature. Similar results were observed with IgE anti-DNP (FIG. 4c) or with IgE anti-ovalbumin (FIG. 10/Supplementary FIG. 4). Anaphylaxis was similarly reduced in mice given L. casei orally for 4 weeks and by gavage during the 4^{th} week (FIG. 4d).

Arthritis was induced by injecting mice with K/B×N serum intravenously at day 0. Inflammation was markedly reduced in mice injected intraperitoneally with L. casei daily between days −2 and +4 (FIG. 5a) and abrogated when injected between days −9 and +2 (FIG. 5b). No protection was observed in mice given L. casei orally for 4 weeks and by gavage during the 4^{th} week (FIG. 5b). Interestingly, L. casei could not only prevent arthritis when administered before K/B×N serum, but also reduce an ongoing arthritis when administered after K/B×N serum (FIG. 5c). Synovitis and neutrophil infiltration observed 6 days after K/B×N serum injection in PBS-treated mice (Kouskoff et al., 1996) was abrogated in mice injected with L. casei i.p. between days −9 and +2 (FIG. 5d).

These three sets of experiments indicate that L. casei can decrease IgE- and IgG-dependent inflammation in mast cell-dependent in vivo models of allergy and autoimmune arthritis.

### Example 6

### L. Casei Inhibits Human Mast Cell and Basophil Activation

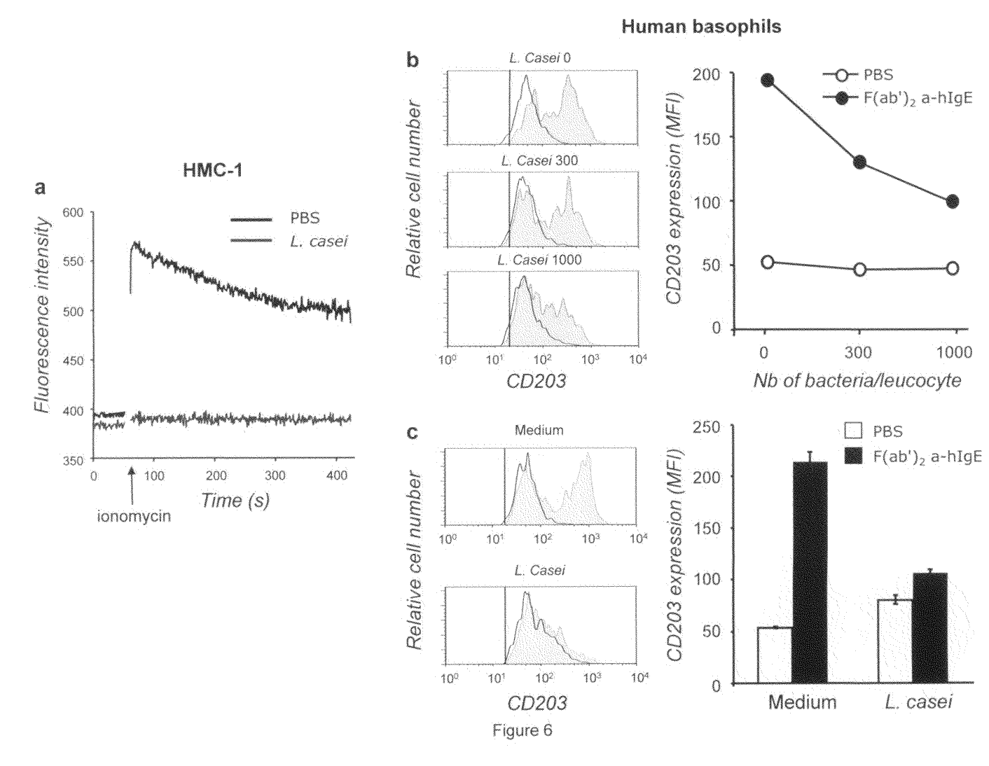

Finally, the inventors investigated whether L. casei could affect human mast cells and basophils. The human mast cell line HMC-1 expresses no FcεRI and contains few granules. An overnight incubation of HMC-1 cells with γ-irradiated L. casei abrogated ionomycin-induced Ca²⁺ responses (FIG. 6a). An overnight incubation of normal white blood cells with γ-irradiated L. casei dose-dependently inhibited anti-IgE-induced CD203c upregulation in basophils (FIGS. 6b & 6c). CD203c upregulation is a marker of basophil activation. L. casei can therefore inhibit human mast cell and basophil activation.

### Example 7

### Discussion

The above results show that L. casei can inhibit the effector phase of immune inflammation by acting on mast cells. This finding provides new insights into the mechanisms by which probiotics might protect from allergies and autoimmune diseases. It has fundamental and clinical implications.

L. casei markedly inhibited IgE-induced mouse mast cell activation when cells and bacteria were incubated overnight before sensitization. Nonpathogenic E. coli strains were previously reported to inhibit mast cell activation in vitro (Kulka et al., 2006; Magerl et al., 2008). E. coli is however not considered as a probiotic. Besides, not all commensal bacteria have the same properties. Indeed, S. thermophilus did not inhibit mast cell activation. Inhibition could be induced either by live or by irradiated L. casei, suggesting that metabolites secreted by live bacteria were not responsible for inhibition. Supporting this assumption, neither high concentrations of lactic acid (not shown) nor supernatant from L. casei incubated overnight in mast cell culture medium (not shown) detectably inhibited mast cell activation. Also, inhibition was abrogated when cells were separated from bacteria by a semi-permeable membrane in transwells. These observations altogether indicate that L. casei needs to be in contact with mast cells for inhibition to occur. Bacteria attached to cells could indeed be observed by video microscopy, but to few mast cells only, after an overnight incubation with L. casei. This could be explained if cell-bacteria contacts were transient and if their effects on individual mast cells lasted longer than contacts. That inhibition required mast cells and bacteria to be incubated for a few hours and increased with the duration of incubation could also be explained if mast cells were individually affected upon contact with bacteria, inhibition becoming detectable when a high enough proportion of cells have encountered bacteria. The inventors could obtain no evidence that phagocytosis was involved in inhibition since mast cells did not detectably internalize irradiated (not shown) or live L. casei.

Since a contact with specific strains of bacteria was required for inhibition, a candidate receptor on mast cells was searched. The inventors failed to identify one such receptor. That inhibition occurred in MyD88^{-/-} cells excludes TLR2, 4, 5, 7 and 9 (O'Neill and Bowie, 2007), and inhibition did occur in TLR2/4^{-/-} cells. We did not study TRIF^{-/-} cells, but inhibition was unaffected in cells lacking TLR4 or TLR3 which signal through TRIF (O'Neill and Bowie, 2007). Inhibition was unaffected either in cells lacking NOD2 which binds muramyl dipeptide and promotes apoptosis (Hsu et al., 2008). None, among the main receptors known to mediate the interactions between cells and microorganisms seems therefore involved in L. casei-induced inhibition of mast cell activation. Other molecules involved in the regulation of intracellular cell signaling were also dispensable (not shown). Indeed, L. casei could inhibit secretory responses of mast cells lacking FcγRIIB which binds viral (Ravanel et al., 1997) and fungal (Monari et al., 2006) molecules, and which negatively regulates cell activation, FcγRIIIA which binds E. coli and can generate inhibitory signals (Pinheiro da Silva et al., 2007), FcRγ which mediates FcR signaling (Takai et al., 1994) or DAP12 which mediates the inhibition of FcR and TLR signaling by TREM-2 (Hamerman et al., 2006). Whether another receptor can, alone, account for inhibition or whether L. casei could use several receptors with similar signaling properties, which could replace each other, remains to be determined.

Inhibition was apparently not due to a possible “desensitization” of mast cells, resulting from a previous activation during the overnight incubation with L. casei. Indeed, L. casei detectably induced neither the release of β-hexosaminidase following a 20-min incubation, nor the secretion of cytokines or chemokines following a 3-h incubation with mast cells, i.e., at time points when these secretory responses were maximum upon stimulation with IgE and antigen. Total β-hexosaminidase content was identical in mast cells incubated overnight with L. casei or without, as well as spontaneous β-hexosaminidase release. Noticeably, L. casei induced no detectable secretion of IL-10 by mast cells. This does not exclude the possible involvement of other inhibitory cytokines, such as TGF-β. Inhibition was not due to a possible cytotoxic effect of L. casei on mast cells, as judged by propidium iodide/annexin V staining, and as confirmed by the reversibility of inhibition. Inhibition was indeed transient and lasted less than 24 h. Inhibition was not due to a possible decreased FcεRI expression or decreased binding of IgE to FcεRI (not shown) as assessed by immunofluorescence. In accordance with this observation, L. casei did not prevent FcεRI aggregation from transducing intracellular signals. L. casei, however, inhibited some, but not all signaling events. Both proximal events, such as Syk, LAT and PLC-Y phosphorylation, and distal events, such as Erk1/2, JNK, and p38 phosphorylation, as well as the Ca²⁺ response were affected. Alterations of distal signals could result from alterations of proximal signals. Noticeably, L. casei selectively inhibited the Lyn/Syk/LAT pathway, leaving both proximal and distal signals of the Fyn/Gab2/PI3K pathway unaffected.

As a consequence of its effects on intracellular signaling, L. casei inhibited mast cell degranulation and the secretion of the four cytokines (IL-5, IL-6, IL-13 and TNF-α) and the two chemokines (MCP-1 and MIP-1α) found to be induced upon FcεRI aggregation using the Luminex technology. Importantly, L. casei also inhibited the release of β-hexosaminidase by mouse mast cells induced by preformed IgG immune complexes made with GPI and K/B×N serum. This indicates that L. casei can inhibit mast cell activation by receptors other than FcεRI. Mast cell activation by IgG immune complexes indeed depends on FcγRIIIA (Hazenbos et al., 1996; Latour et al., 1992). It also indicates that L. casei can inhibit the secretory responses of mast cells other than BMMC. Indeed, PCMC were used for these experiments, instead of BMMC, which respond poorly to IgG stimulations. PCMC are a model of mature serosal-type mast cells (Malbec et al., 2007) that better account for allergic and autoimmune inflammation, which occur in tissues, than BMMC which are immature cells, the physiologic counterparts of which are unknown.

To determine the possible in vivo relevance of the effects of L. casei observed on mast cells in vitro, a model of adoptive systemic anaphylaxis in normal mice injected i.v. with IgE-sensitized mast cells was devised. Lethal adoptive anaphylaxis induced upon antigen challenge in mice injected with IgE-sensitized BMMC, was abrogated in mice injected with BMMC incubated with L. casei prior sensitization with IgE. This observation confirms that mast cells are sufficient for fatal IgE-induced anaphylaxis, and it indicates that the inhibition observed by measuring β-hexosaminidase and TNF-α in mast cell supernatants affects all mediators involved in anaphylaxis. Importantly, L. casei could also inhibit not only IgE-induced passive systemic anaphylaxis, but also IgG-induced passive arthritis, when administered i.p. As these models of allergic and autoimmune diseases bypass the induction phase of the immune response, these results indicate that L. casei can inhibit the effector phase of immune inflammation.

Cells other than mast cells concur to passive anaphylaxis and arthritis. These include basophils, neutrophils, platelets and macrophages. How the bacteria could reach these cells is indeed intriguing, if the requirement for a contact between cells and bacteria observed in vitro applies in vivo. L. casei was injected i.p. in the above-described experimental settings. Whether it could exit the peritoneal cavity and reach other tissues is not known. Mast cells are present in every tissue, but the location of mast cells contributing to systemic anaphylaxis or to arthritis is not known. Normally, commensals reside in the gut. Bacteria can be taken up by dendritic (Niess et al., 2005) or M cells (Kerneis et al., 1997) and cross the gut epithelium, but in low numbers. Also, bacteria are transiently found in blood, especially after meals. It is possible that these processes can bring L. casei close to mast cells and/or inflammatory cells under physiological or pathological conditions.

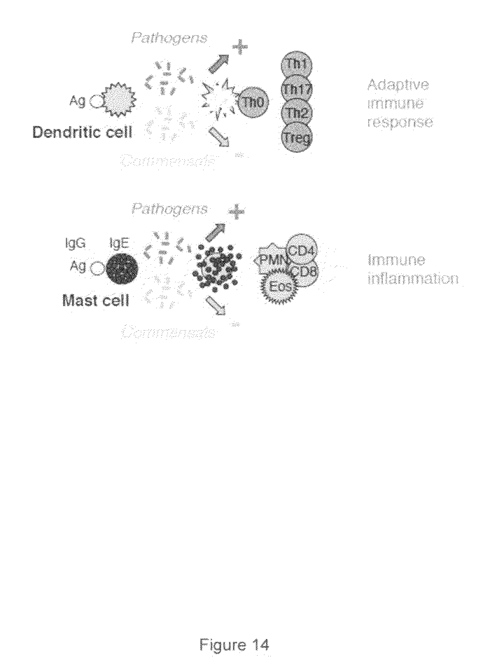

The above findings have the following three fundamental and practical implications. First, dendritic cells are activated by pathogen products, leading to their maturation and enabling them to present antigen to T cells (Steinman and Hemmi, 2006). Probiotics, including L. casei, modulate dendritic cell differentiation. Likewise, mast cells can be activated by pathogens and they contribute to the protection against infections (Bischoff and Kramer, 2007; Echtenacher et al., 1996; Thakurdas et al., 2007). It is herein shown that L. casei markedly inhibits the activation of mast cells and dampens their pro-inflammatory effects. The positive and negative effects of microorganisms on mast cell activation in the effector phase of immune responses is reminiscent of positive and negative effects of microorganisms on dendritic cell activation in the afferent phase of immune responses (FIG. 14). Second, the hygiene hypothesis (Yazdanbakhsh et al., 2002) was proposed to account for the allergy “epidemics” that has been expanding over the last decades (Holgate, 1999). According to this hypothesis, a lower exposure to pathogens resulting from a better hygiene of life, the generalization of vaccination and an extensive use of antibiotics, favored the development of Th2 responses instead of Th1 responses (Oyama et al., 2001). The role assigned to microorganisms in the hygiene hypothesis has been based mostly on their effects on the control of Th1/Th2 polarization by dendritic cells (Segawa et al., 2008; So et al., 2008). Not only Th2 diseases, such as allergies, but also Th1 diseases, such as type-1 diabetes, Crohn's disease and multiple sclerosis have however increased in parallel (Bach, 2002). A common feature of these two types of diseases is inflammation, which depends on mast cells. The inhibitory effects of a nonpathogenic bacterium on mast cell activation described here strengthen the hygiene hypothesis by providing a mechanisms which could possibly account for the parallel increased incidence of Th1- and Th2-type inflammatory immune diseases. Most importantly, the above findings have an important practical consequence. If it can inhibit not only the induction of immune responses leading to the generation of anti-allergen IgE antibodies or IgG autoantibodies, but also the effector phase of allergic or autoimmune inflammation, L. casei is appropriate for preventing symptoms in allergic or autoimmune patients who have already produced specific IgE or autoantibodies.

### REFERENCES

Alexopoulou, L., Holt, A. C., Medzhitov, R. and Flavell, R. A. (2001) Recognition of double-stranded RNA and activation of NF-kappaB by Toll-like receptor 3. Nature, 413, 732-738.
Bach, J. F. (2002) The effect of infections on susceptibility to autoimmune and allergic diseases. N Engl J Med, 347, 911-920.
Barreau, F., Meinzer, U., Chareyre, F., Berrebi, D., Niwa-Kawakita, M., Dussaillant, M., Foligne, B., Ollendorff, V., Heyman, M., Bonacorsi, S., Lesuffleur, T., Sterkers, G., Giovannini, M. and Hugot, J. P. (2007) CARD15/NOD2 is required for Peyer's patches homeostasis in mice. PLoS One, 2, e523.
Bischoff, S. C. and Kramer, S. (2007) Human mast cells, bacteria, and intestinal immunity. Immunol Rev, 217, 329-337.
Bruhns, P., Samuelsson, A., Pollard, J. W. and Ravetch, J. V. (2003) Colony-stimulating factor-1-dependent macrophages are responsible for IVIG protection in antibody-induced autoimmune disease. Immunity, 18, 573-581.
Echtenacher, B., Mannel, D. N. and Hultner, L. (1996) Critical protective role of mast cells in a model of acute septic peritonitis. Nature, 381, 75-77.
Hamada, K., Suzaki, Y., Goldman, A., Ning, Y. Y., Goldsmith, C., Palecanda, A., Coull, B., Hubeau, C. and Kobzik, L. (2003) Allergen-independent maternal transmission of asthma susceptibility. J Immunol, 170, 1683-1689.
Hamerman, J. A., Jarjoura, J. R., Humphrey, M. B., Nakamura, M. C., Seaman, W. E. and Lanier, L. L. (2006) Cutting edge: inhibition of TLR and FcR responses in macrophages by triggering receptor expressed on myeloid cells (TREM)-2 and DAP12. J Immunol, 177, 2051-2055.
Hazenbos, W. L., Gessner, J. E., Hofhuis, F. M., Kuipers, H., Meyer, D., Heijnen, I. A., Schmidt, R. E., Sandor, M., Capel, P. J., Daeron, M., van de Winkel, J. G. and Verbeek, J. S. (1996) Impaired IgG-dependent anaphylaxis and Arthus reaction in Fc gamma RIII (CD16) deficient mice. Immunity, 5, 181-188.
Holgate, S. T. (1999) The epidemic of allergy and asthma. Nature, 402, B2-4.
Hsu, L. C., Ali, S. R., McGillivray, S., Tseng, P. H., Mariathasan, S., Humke, E. W., Eckmann, L., Powell, J. J., Nizet, V., Dixit, V. M. and Karin, M. (2008) A NOD2-NALP1 complex mediates caspase-1-dependent IL-1beta secretion in response to Bacillus anthracis infection and muramyl dipeptide. Proc Natl Acad Sci USA, 105, 7803-7808.
Kawai, T., Adachi, O., Ogawa, T., Takeda, K. and Akira, S. (1999) Unresponsiveness of MyD88-deficient mice to endotoxin. Immunity, 11, 115-122.
Kemeis, S., Bogdanova, A., Kraehenbuhl, J. P. and Pringault, E. (1997) Conversion by Peyer's patch lymphocytes of human enterocytes into M cells that transport bacteria. Science, 277, 949-952.
Kouskoff, V., Korganow, A. S., Duchatelle, V., Degott, C., Benoist, C. and Mathis, D. (1996) Organ-specific disease provoked by systemic autoimmunity. Cell, 87, 811-822.
Kulka, M., Fukuishi, N., Rottem, M., Mekori, Y. A. and Metcalfe, D. D. (2006) Mast cells, which interact with Escherichia coli, up-regulate genes associated with innate immunity and become less responsive to Fc(epsilon)RI-mediated activation. J Leukoc Biol, 79, 339-350.
Latour, S., Bonnerot, C., Fridman, W. H. and Daeron, M. (1992) Induction of tumor necrosis factor-alpha production by mast cells via Fc gamma R. Role of the Fc gamma RIII gamma subunit. J Immunol, 149, 2155-2162.
Lee, D. M., Friend, D. S., Gurish, M. F., Benoist, C., Mathis, D. and Brenner, M. B. (2002) Mast cells: a cellular link between autoantibodies and inflammatory arthritis. Science, 297, 1689-1692.
Liu, F. T., Bohn, J. W., Ferry, E. L., Yamamoto, H., Molinaro, C. A., Sherman, L. A., Klinman, N. R. and Katz, D. H. (1980) Monoclonal dinitrophenyl-specific murine IgE antibody: preparation, isolation, and characterization. J Immunol, 124, 2728-2737.
Magerl, M., Lammel, V., Siebenhaar, F., Zuberbier, T., Metz, M. and Maurer, M. (2008) Non-pathogenic commensal Escherichia coli bacteria can inhibit degranulation of mast cells. Exp Dermatol, 17, 427-435.
Malbec, O., Roget, K., Schiffer, C., lannascoli, B., Dumas, A. R., Arock, M. and Daeron, M. (2007) Peritoneal cell-derived mast cells: an in vitro model of mature serosal-type mouse mast cells. J Immunol, 178, 6465-6475.
Monari, C., Kozel, T. R., Paganelli, F., Pericolini, E., Perito, S., Bistoni, F., Casadevall, A. and Vecchiarelli, A. (2006) Microbial immune suppression mediated by direct engagement of inhibitory Fc receptor. J Immunol, 177, 6842-6851.

Niess, J. H., Brand, S., Gu, X., Landsman, L., Jung, S., McCormick, B. A., Vyas, J. M., Boes, M., Ploegh, H. L., Fox, J. G., Littman, D. R. and Reinecker, H. C. (2005) CX3CR1-mediated dendritic cell access to the intestinal lumen and bacterial clearance. Science, 307, 254-258.
O'Neill, L. A. and Bowie, A. G. (2007) The family of five: TIR-domain-containing adaptors in Toll-like receptor signalling. Nat Rev Immunol, 7, 353-364.
Oyama, N., Sudo, N., Sogawa, H. and Kubo, C. (2001) Antibiotic use during infancy promotes a shift in the T(H)1/T(H)2 balance toward T(H)2-dominant immunity in mice. J Allergy Clin Immunol, 107, 153-159.
Pinheiro da Silva, F., Aloulou, M., Skurnik, D., Benhamou, M., Andremont, A., Velasco, I. T., Chiamolera, M., Verbeek, J. S., Launay, P. and Monteiro, R. C. (2007) CD16 promotes Escherichia coli sepsis through an FcR gamma inhibitory pathway that prevents phagocytosis and facilitates inflammation. Nat Med, 13, 1368-1374.

Ramphal, R., Balloy, V., Huerre, M., Si-Tahar, M. and Chignard, M. (2005) TLRs 2 and 4 are not involved in hypersusceptibility to acute Pseudomonas aeruginosa lung infections. J Immunol, 175, 3927-3934.
Ravanel, K., Castelle, C., Defrance, T., Wild, T. F., Charron, D., Lotteau, V. and Rabourdin-Combe, C. (1997) Measles virus nucleocapsid protein binds to FcgammaRII and inhibits human B cell antibody production. J Exp Med, 186, 269-278.
Segawa, S., Nakakita, Y., Takata, Y., Wakita, Y., Kaneko, T., Kaneda, H., Watari, J. and Yasui, H. (2008) Effect of oral administration of heat-killed Lactobacillus brevis SBC8803 on total and ovalbumin-specific immunoglobulin E production through the improvement of Th1/Th2 balance. Int J Food Microbiol, 121, 1-10.
So, J. S., Kwon, H. K., Lee, C. G., Yi, H. J., Park, J. A., Lim, S. Y., Hwang, K. C., Jeon, Y. H. and Im, S. H. (2008) Lactobacillus casei suppresses experimental arthritis by down-regulating T helper 1 effector functions. Mol Immunol, 45, 2690-2699.
Steinman, R. M. and Hemmi, H. (2006) Dendritic cells: translating innate to adaptive immunity. Curr Top Microbiol Immunol, 311, 17-58.
Takai, T., Li, M., Sylvestre, D., Clynes, R. and Ravetch, J. V. (1994) FcR gamma chain deletion results in pleiotrophic effector cell defects. Cell, 76, 519-529.
Thakurdas, S. M., Melicoff, E., Sansores-Garcia, L., Moreira, D. C., Petrova, Y., Stevens, R. L. and Adachi, R. (2007) The mast cell-restricted tryptase mMCP-6 has a critical immunoprotective role in bacterial infections. J Biol Chem, 282, 20809-20815.
Yazdanbakhsh, M., Kremsner, P. G. and van Ree, R. (2002) Allergy, parasites, and the hygiene hypothesis. Science, 296, 490-494.

1. A method for inhibiting mast cell activation comprising administering to a subject in need thereof an effective amount of a L. casei strain or bacterial lysate or a mast cell inhibiting fraction of said bacterial lysate.

2. The method of claim 1, wherein said subject is in need of preventing, suppressing or inhibiting IgE-induced mast cell activation.

3. The method of claim 1, wherein said subject is in need of preventing, suppressing, or inhibiting IgG-induced mast cell activation.

4. The method of claim 1, wherein said subject is in need of preventing, alleviating or treating a disease or a disorder involving mast cell activation.

5. The method of claim 1, wherein said subject is in need of preventing, alleviating or treating an allergy.

6. The method of claim 1, wherein said subject is in need of preventing, alleviating or treating an autoimmune disease.

7. The method of claim 4, wherein said disorder or disease is an inflammatory disorder.

8. The method of claim 4, wherein said disorder or disease is arthritis.

9. The method of claim 1, wherein said L. casei strain is a L. casei ssp. Paracasei strain.

10. The method of claim 1, wherein said L. casei strain is the strain deposited at the CNCM on Dec. 30, 1994, under the number I-1518.

11. The method of claim 1, wherein the L. casei as formulated as a medicinal product.

12. The method of claim 1, wherein said L. casei bacteria are used alive.

13. The method of claim 1, wherein said L. casei bacteria are used as whole dead cells.

14. The method of claim 1 wherein the L. casei are in the form of a bacterial lysate or a bacterial fraction of said lysate.

15. The method according to claim 1, comprising administering a composition containing L. casei which is formulated so that its uptake leads to a direct contact between mast cells and the bacteria, bacterial lysate and/or bacterial fraction comprised in said composition.

16. A screening process for identifying a bacterial composition which prevents or treats a disease or disorder mediated by mast cells, comprising:
a) administering an amount of said bacterial composition to a normal mouse by parenteral route,
b) subsequently injecting intravenously the serum from an autoimmune K/B×N mouse; and
c) determining the severity of arthritis in said normal mouse, optionally by analyzing the score of arthritis by clinical examination.

17. A process of screening for identifying a bacterial composition which treats or prevents a mast cell-dependent allergic process comprising:
a) administering orally or parenterally of a said bacterial composition to normal mice,
b) subsequently injecting intravenously said normal mice with purified IgE or serum containing IgE antibodies specific for an allergen,
c) challenging intravenously said IgE-injected mice, optionally one or more days later, with said allergen,
e) determining the intensity of anaphylactic shock induced by said challenge, optionally by measuring a decrease in body temperature,
wherein a reduced anaphylactic shock compared to control mice not administered said bacterial composition is indicative of a bacterial composition which treats or prevents a mast cell-dependent allergic process.

18. The process of claim 16, wherein said bacterial composition comprises L. casei cells or components thereof.

19. An animal model for anaphylaxis reaction comprising:
a) sensitizing in vitro mast cells by contacting them with IgE specific for an allergen of interest,
b) injecting the sensitized mast cells to normal mice, subsequently
c) injecting into said mice the allergen of interest.

20. A process for identifying or screening a molecule which decreases the anaphylactic reaction in vivo, comprising:
a) pre-incubating mast cells with a molecule of interest,
b) sensitizing in vitro mast cells by contacting them with IgE specific for an allergen of interest,
c) injecting the sensitized mast cells to normal mice, subsequently
d) injecting into said mice the allergen of interest,
e) monitoring the anaphylactic shock, optionally by measuring the body temperature of said mice injected with the allergen of interest;
wherein a reduced amount of anaphylactic reaction in the treated mice compared to control mice administered mast cells not treated with said molecule of interest indicative of molecule which decreases the anaphylactic reaction in vivo.

21. The process of claim 20, wherein said molecule of interest is a component of Lactobacillus.

22. A method for making a composition for inhibiting mast cell activation comprising admixing an effective concentration or amount of L. casei or bacterial lysate of L. casei or a mast cell inhibiting fraction of said bacterial lysate, with at least one physiologically or pharmacologically acceptable liquid, semi-solid, or solid carrier, excipient or buffer, food or food component, animal feed or animal feed component.

23. A method for inhibiting mast cell activation comprising contacting a mast cell with L. casei, or a bacterial lysate of L. casei or a mast cell inhibiting fraction of said bacterial lysate or contacting the mast cell with a composition made by the method of claim 22.
